(19) United States
(12) Patent Application Publication
(10) Pub. No.: US 2008/0021070 A1
(43) Pub. Date: Jan. 24, 2008

(54) PYRIDINE DERIVATIVES, PREPARATION AND THERAPEUTIC APPLICATION THEREOF

(75) Inventors: Lionel Barre, Montbazin (FR); Francis Barth, Saint Georges D'Orques (FR); Christian Congy, Saint Gely Du Fesc (FR); Murielle Rinaldi-Carmona, Saint Georges D'Orques (FR); Philippe Pointeau, Sain Clement De Riviere (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 11/695,770

(22) Filed: Apr. 3, 2007

Related U.S. Application Data
(63) Continuation of application No. PCT/FR05/02566, filed on Oct. 17, 2005.

(30) Foreign Application Priority Data
Oct. 18, 2004 (FR) 0411030

(51) Int. Cl. A61K 31/4427; A61K 31/44; A61K 31/4433; A61K 31/4436; A61K 31/4439; A61P 25/00; A61P 29/00; A61P 3/00; A61P 37/00; C07D 213/56; C07D 401/12; C07D 405/12; C07D 409/12

(52) U.S. Cl. 514/339; 514/336; 514/343; 514/357; 546/278.1; 546/278.4; 546/280.4; 546/281.7; 546/337

(57) ABSTRACT

The invention relates to compounds of formula (I): 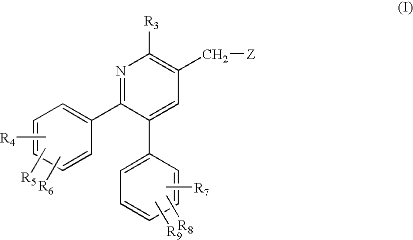
Wherein Z, R₃ to R₉ are as described herein. The invention also relates to methods of preparation of compounds of formula (I) and intermediates thereof. The compounds of this invention are active at CB1 cannabinoid receptor site, and are therefore, useful as pharmaceutical agents in treating a variety of diseases caused by the effects of CB1 cannabinoid receptors.

### CROSS REFERENCE TO RELATED APPLICATIONS

This application is a continuation of International application No. PCT/FR2005/002,566, filed Oct. 17, 2005, which is incorporated herein by reference in its entirety; which claims the benefit of priority of French Patent Application No. 04/11,030, filed Oct. 18, 2004.

### BACKGROUND OF THE INVENTION

1. Field of the Invention

The present invention relates to pyridine derivatives, to their preparation and to their therapeutic application.

2. Description of the Art

International patent application WO 03/082 191 describes pyridine derivatives of formula: 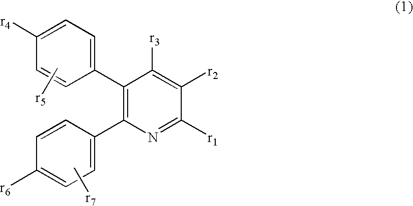
in which the substituents r₁ to r₇ have different values.

Patent application WO 2002/055 502 describes compounds of formula:

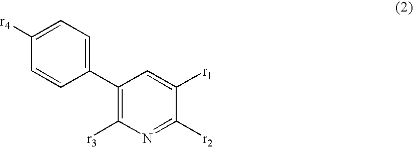

Both of the aforementioned references are incorporated herein by reference in their entirety.

### SUMMARY OF THE INVENTION

The subject of the present invention is compounds corresponding to the formula: 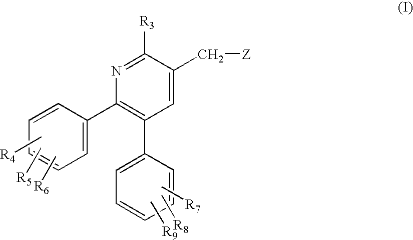
in which
Z represents a group N(R₁)XR₂, N(R₁)COOR′₂ or OCON(R₁)R′₂;
X represents a group —CO—, —SO₂—, —CON(R₁₀)— or —CSN(R₁₀)—;
R₁ represents a hydrogen atom or a (C₁-C₄)alkyl group;
R₂ represents:

a (C₃-C₁₀)alkyl group, which is unsubstituted or substituted with a CF₃ group;

a non-aromatic (C₃-C₁₂) carbocyclic radical, which is unsubstituted or substituted one or more times with identical or different substituents chosen from a (C₁-C₄)alkyl, hydroxyl, (C₁-C₄)alkoxy, (C₁-C₄)alkylthio or cyano group;

a saturated or unsaturated heterocyclic radical of 4 to 8 atoms containing oxygen, sulfur or nitrogen, which is unsubstituted or substituted with one or more identical or different substituents chosen from a halogen atom and a (C₁-C₄)alkyl, hydroxyl, trifluoromethyl, (C₁-C₄)alkoxy, trifluoromethoxy, (C₁-C₄)alkylthio, cyano or nitro group;

an indolyl group, which is unsubstituted or substituted with a halogen atom or with a (C₁-C₄)alkyl, trifluoromethyl, hydroxyl, (C₁-C₄)alkoxy, trifluoromethoxy, (C₁-C₄)alkyl-thio, cyano or nitro group;

a tetrahydro-1- or -2-naphthyl; a 1- or 2-naphthyl;

a benzothiophenyl or a benzofuryl;

a phenyl, which is unsubstituted or substituted one or more times with identical or different substituents chosen from a halogen atom, a (C₁-C₄)alkyl, trifluoromethyl, trifluoromethoxy, hydroxyl, (C₁-C₄)alkoxy, cyano, nitro, (C₁-C₄)alkanoyl or phenyl group and a group S(O)ₙAlk or NR₁₃R₁₄;

a benzodioxyl;

a phenoxymethyl, a 1-phenoxyethyl or a 1-methyl-1-phenoxyethyl, the phenyl groups being unsubstituted or substituted one or more times with identical or different substituents chosen from a halogen atom and a (C₁-C₄)alkyl or trifluoromethyl group;

a phenylcyclopropyl, the phenyl group being unsubstituted or substituted one or more times with identical or different substituents chosen from a halogen atom and a (C₁-C₄)alkyl or trifluoromethyl group;

a (C₁-C₂)alkylene substituted with one or two identical or different substituents chosen from:
(i) a (C₁-C₄)alkyl group;
(ii) a non-aromatic C₃-C₁₂ carbocyclic radical, which is unsubstituted or substituted one or more times with a (C₁-C₄)alkyl group;
(iii) a phenyl, which is unsubstituted or substituted with one or more substituents, which may be identical or different, chosen from a halogen atom, a (C₁-C₄)alkyl, hydroxyl, trifluoromethyl, (C₁-C₄)alkoxy, trifluoromethoxy, (C₁-C₄)alkanoyl, cyano, nitro or phenyl group and a group S(O)ₙAlk or NR₁₃R₁₄;
(iv) a saturated or unsaturated heterocyclic radical of 4 to 8 atoms, containing oxygen, sulfur or nitrogen, which is unsubstituted or substituted with one or more substituents, which may be identical or different, chosen from a halogen atom and a (C₁-C₄)alkyl or trifluoromethyl group;

furthermore, when X represents a group —CON(R₁₀)— or —CSN(R₁₀)—, R₂ may represent a (C₁-C₆)alkanoyl group or a benzoyl or benzylcarbonyl group, the phenyl group of the said groups being unsubstituted or substituted with identical or different substituents chosen from a halogen atom and a (C₁-C₄)alkyl or trifluoromethyl group;

R′₂ represents a phenyl, which is unsubstituted or substituted one or more times with identical or different substituents chosen from a halogen atom and a (C₁-C₄)alkyl, trifluoromethyl, cyano, nitro or (C₁-C₄)alkoxy group;

R₃ represents a hydrogen atom or a (C₁-C₄)alkyl, cyano, (C₁-C₄)alkoxymethyl or hydroxymethyl group;

R₄, R₅, R₆, R₇, R₈ and R₉ represent, independently of each other, a hydrogen or halogen atom, a (C₁-C₆)alkyl, (C₁-C₆)alkoxy, trifluoromethyl, trifluoromethoxy, cyano or nitro group or a group S(O)ₙAlk;

R₁₀ represents a hydrogen atom or a (C₁-C₄)alkyl group;

or R₂ and R₁₀, together with the nitrogen atom to which they are attached, constitute a heterocyclic radical of 4 to 8 atoms, possibly containing a second hetero atom chosen from an oxygen, a sulfur and a nitrogen atom, which is unsubstituted or substituted one or more times with a (C₁-C₄)alkyl group; a (C₁-C₄)alkanoyl group; a group NR₁₁R₁₂ or CONR₁₁R₁₂; a phenyl group, which is unsubstituted or substituted one or more times with a halogen atom or a (C₁-C₄)alkyl, (C₁-C₄)alkoxy or trifluoromethyl group;

R₁₁ and R₁₂ represent, independently of each other, a hydrogen atom or a (C₁-C₄)alkyl group, or R₁₁ and R₁₂, together with the nitrogen atom to which they are attached, constitute a heterocyclic radical of 4 to 8 atoms;

n represents 0, 1 or 2;

R₁₃ and R₁₄ represent, independently of each other, a hydrogen atom or a (C₁-C₄)alkyl group, or R₁₃ and R₁₄, together with the nitrogen atom to which they are attached, constitute a saturated or unsaturated heterocyclic radical of 4 to 8 atoms;

Alk represents a (C₁-C₄)alkyl group;
on condition that one of the substituents R₁, R₃, R₅, R₆, R₈ and R₉ is other than hydrogen when R₄ and R₇ simultaneously represent a 4-methoxy group;
in base or addition-salt form, and also in hydrate or solvate form.

### DETAILED DESCRIPTION OF THE INVENTION

More particularly, the present invention relates to the compounds of formula: 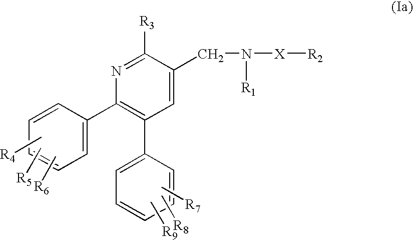
in which

X represents a —CO—, —SO₂— or —CON(R₁₀)— group;

R₁ represents a hydrogen atom or a (C₁-C₄)alkyl group;

R₂ represents:

a (C₃-C₁₀)alkyl group;

a non-aromatic (C₃-C₁₂) carbocyclic radical, which is unsubstituted or substituted one or more times with a (C₁-C₄)alkyl group;

an indolyl, which is unsubstituted or substituted on the nitrogen atom with a (C₁-C₄)alkyl;

a phenyl, which is unsubstituted or substituted one or more times with identical or different substituents chosen from a halogen atom and a (C₁-C₄)alkyl, trifluoromethyl, trifluoromethoxy, (C₁-C₄)alkoxy, cyano, (C₁-C₄)alkanoyl or phenyl group;

a benzyl, which is unsubstituted or substituted one or more times with identical or different substituents chosen from a halogen atom and a (C₁-C₄)alkyl, trifluoromethyl, (C₁-C₄)alkoxy, cyano or phenyl group;

R₃ represents a hydrogen atom or a (C₁-C₄)alkyl, cyano or (C₁-C₄)alkoxymethylene group;

R₄, R₅, R₆, R₇, R₈ and R₉ represent, independently of each other, a hydrogen or halogen atom, a (C₁-C₆)alkyl, (C₁-C₆)alkoxy or trifluoromethyl group or a group S(O)ₙAlk;

R₁₀ represents a hydrogen atom or a (C₁-C₄)alkyl group;

or R₂ and R₁₀, together with the nitrogen atom to which they are attached, constitute a heterocyclic radical of 4 to 8 atoms, possibly containing a second heteroatom chosen from an oxygen, a sulfur and a nitrogen atom, which is unsubstituted or substituted one or more times with a (C₁-C₄)alkyl group; a (C₁-C₄)alkanoyl group; a group NR₁₁R₁₂ or CONR₁₁R₁₂; a phenyl group, which is unsubstituted or substituted one or more times with a halogen atom or a (C₁-C₄)alkyl, (C₁-C₄)alkoxy or trifluoromethyl group;

R₁₁ and R₁₂ represent, independently of each other, a hydrogen atom or a (C₁-C₄)alkyl group, or R₁₁ and R₁₂, together with the nitrogen atom to which they are attached, constitute a heterocyclic radical of 4 to 8 atoms;

n represents 0, 1 or 2;

Alk represents a (C₁-C₄)alkyl group;
on condition that one of the substituents R₁, R₃, R₅, R₆, R₈ and R₉ is other than hydrogen when R₄ and R₇ simultaneously represent a 4-methoxy group.

The compounds of formula (I) may comprise one or more asymmetric carbon atoms. They may thus exist in the form of enantiomers or diastereoisomers. These enantiomers and diastereoisomers, and also mixtures thereof, including racemic mixtures, form part of the invention.

The compounds of formula (I) may exist in the form of bases or of acid-addition salts. Such addition salts form part of the invention.

These salts may be prepared with pharmaceutically acceptable acids, but the salts of other acids that are useful, for example, for purifying or isolating the compounds of formula (I) also form part of the invention.

The compounds of formula (I) may also exist in the form of hydrates or solvates, i.e. in the form of associations or combinations with one or more water molecules or with a solvent. Such hydrates and solvates also form part of the invention.

In the context of the present invention, the following definitions apply:

a halogen atom: a fluorine, a chlorine, a bromine or an iodine;

a (C₁-C₄)alkyl, (C₁-C₆)alkyl or (C₃-C₁₀)alkyl group, respectively: a linear or branched saturated aliphatic (C₁-C₄), (C₁-C₆) or (C₃-C₁₀) group, respectively. Examples that may be mentioned include methyl, ethyl, propyl, isopropyl, butyl, isobutyl, tert-butyl, pentyl, hexyl, 1-ethylpropyl, 1-propylbutyl, etc. groups;

a (C₁-C₄)alkoxy group: an O-alkyl radical in which the alkyl group is as defined above.

The non-aromatic C₃-C₁₂ carbocyclic radicals comprise fused or bridged monocyclic or polycyclic radicals. The monocyclic radicals include cycloalkyls, for example cyclopropyl, cyclobutyl, cyclopentyl, cyclohexyl, cycloheptyl or cyclooctyl; cyclohexyl and cyclopentyl being preferred. The fused, bridged or spirane bicyclic or tricyclic radicals include, for example, norbornyl, bornyl, isobornyl, noradamantyl, adamantyl, spiro[5.5]undecanyl, bicyclo[2.2.1]heptyl, bicyclo[3.2.1]octyl and bicyclo[3.1.1]heptyl radicals.

The heterocyclic radicals of 4 to 8 atoms comprise azetidinyl, pyrrolidinyl, pyrrolyl, piperidyl, perhydroazepinyl and perhydroazocinyl radicals; the radicals also containing a second hetero atom chosen from an oxygen, a sulfur and a nitrogen atom also comprise imidazolidinyl, pyrazolidinyl, piperazinyl, morpholinyl, thiomorpholinyl, etc. radicals.

Among the compounds of formula (I) that are the subject of the invention, the following are distinguished:
the compounds of formulae (IA), (IB), (IC) and (ID) in which Z represents a group N(R₁)XR₂ and
   either X represents a —CO-group and the substituents R₁ to R₉ are as defined above for the compounds for formula (I): the compounds for formula (IA);
   or X represents an —SO₂-group and the substituents R₁ to R₉ are as defined above for the compounds of formula (I): the compounds of formula (IB);
   or X represents a group —CONR₁₀— and the substituents R₁ to R₁₀ are as defined above for the compounds of formula (I): the compounds of formula (IC);
   or X represents a group —CSNR₁₀ and the substituents R₁ and R₁₀ are as defined above for the compounds of formula (I): the compounds of formula (ID);
the compounds of formula (IE) in which Z represents a group N(R₁)COOR′₂ and the substituents R₁ to R₉ are as defined above for the compounds of formula (I);
the compounds of formula (IF) in which Z represents a group OCO(R₁)R′₂ and the substituents R₁ to R₉ are as defined above for the compounds of formula (I).

Among the compounds of formula (I) that are subjects of the invention, one group of compounds consists of the compounds for which:

Z represents a group N(R₁)XR₂ and X has one of the values defined for (I);

R₁ represents a hydrogen atom;

and/or R₂ represents a 1-propylbutyl or a 2-indolyl, which is unsubstituted or substituted with a (C₁-C₄)alkyl group, or R₂ represents a phenyl, which is unsubstituted or substituted one or more times with identical or different substituents chosen from a halogen atom and a (C₁-C₄)alkyl, trifluoromethyl, (C₁-C₄)alkoxy, cyano or phenyl group;

and/or R₃ represents a methyl or methoxymethyl group;

and/or R₄ represents a chlorine or bromine atom or a methoxy group;

and/or R₇ and R₈ each represent a chlorine atom;

and/or R₅, R₆ and R₉ represent hydrogen;

in base or addition-salt form and also in hydrate or solvate form.

Most particularly, the compounds of formula (IA) that are distinguished are those in which:

Z represents a group NHCOR₂;

R₂ represents a 1-propylbutyl group, an indolyl group, which is unsubstituted or substituted with a (C₁-C₄)alkyl group, a phenyl group, which is unsubstituted or substituted with a halogen atom or a trifluoromethyl;

R₃ represents a methyl or methoxymethyl group;

R₄ represents a chlorine or bromine atom or a methoxy group;

R₇ and R₈ each represent a chlorine atom;

R₅, R₆ and R₉ represent hydrogen;

in base or addition-salt form, and also in hydrate or solvate form.

The compounds of formula (IB) that are also distinguished are those in which:

Z represents a group NHSO₂R₂;

R₂ represents a phenyl group, which is unsubstituted or substituted with a halogen atom or with a trifluoromethyl group;

R₃ represents a methyl or methoxymethyl group;

R₄ represents a chlorine or bromine atom or a methoxy group;

R₇ and R₈ each represent a chlorine atom;

R₅, R₆ and R₉ represent hydrogen;

in base or addition-salt form and also in hydrate or solvate form.

Among the described compounds of the invention that may especially be mentioned are the following compounds:
N-{[-6-(4-chlorophenyl)-5-(2,4-dichlorophenyl)-2-methylpyridin-3-yl]methyl}-1H-indole-2-carboxamide.
N-{[-6-(4-chlorophenyl)-5-(2,4-dichlorophenyl)-2-methylpyridin-3-yl]methyl}-4-(trifluoromethyl)benzenesulfonamide.
N-{[-6-(4-chlorophenyl)-5-(2,4-dichlorophenyl)-2-methylpyridin-3-yl]methyl-}-N′-4-(trifluoromethyl)phenyl]urea.
N-{[5-(2,4-dichlorophenyl)-2-(methoxymethyl)-6-(4-methoxyphenyl)pyridin-3-yl]methyl}-2-propylpentanamide.
N-{[5-(2,4-dichlorophenyl)-2-(methoxymethyl)-6-(4-methoxyphenyl)pyridin-3-yl]methyl}-1H-indole-2-carboxamide.
N-{[6-(4-chlorophenyl)-5-(2,4-dichlorophenyl)-2-(methoxymethyl)pyridin-3-yl]methyl}-4-(trifluoromethoxy)benzamide.
N-{[6-(4-chlorophenyl)-5-(2,4-dichlorophenyl)-2-(methoxymethyl)pyridin-3-yl]methyl}-2-propylpentanamide.
   in base or addition-salt form and also in hydrate or solvate form.

In the text hereinbelow, the term “protecting group Pg” means a group that makes it possible firstly to protect a reactive function such as a hydroxyl or an amine during a synthesis, and secondly to regenerate the intact reactive function at the end of the synthesis. Examples of protecting groups and also protection and deprotection methods are given in “Protective Groups in Organic Synthesis”, Greene et al., 2^{nd} Edition (John Wiley & Sons, Inc., New York), 1991.

In the text hereinbelow, the term “leaving group” means a group that may be readily cleaved from a molecule by breaking a heterolytic bond, with loss of an electron pair. This group may thus be readily replaced with another group during a substitution reaction, for example. Such leaving groups are, for example, halogens or an activated hydroxyl group such as a methane sulfonate, benzene sulfonate, p-toluene sulfonate, triflate (or trifluoromethane sulfonate), acetate, etc. Examples of leaving groups and references for preparing them are given in “Advances in Organic Chemistry”, J. March, 3^{rd} Edition, Wiley Interscience, 1985, p. 310-316.

In accordance with the invention, the compounds of general formula (I) in which Z represents a group N(R₁)XR₂ or N(R₁)COOR′₂ may be prepared according to the process characterized in that a compound of formula: 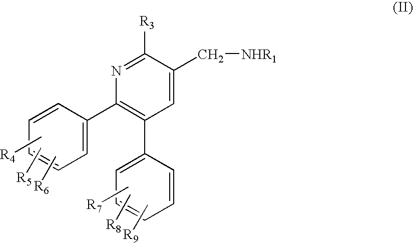
in which the substituents R₁ and R₃ to R₉ are as defined for (I), is treated:

either with an acid of formula R₂CO₂H (III) in which R₂ is as defined for (I), or with an activated derivative of the said acid, when a compound of formula (IA) in which X represents a —CO-group is to be prepared;

or with a sulfonyl halide of formula R₂SO₂Hal (IV) in which R₂ is as defined for (I) and Hal represents a halogen atom, preferentially chlorine, when a compound of formula (IB) in which X represents an —SO₂-group is to be prepared;

or an aryloxycarbonyl halide of formula HalCOOR′₂ in which R′₂ is as defined for a compound of formula (I), when a compound of formula (IE) in which Z represents a group N(R₁)COOR′₂ is to be prepared;

or with an isocyanate of formula R₂—N═C═O (VII) in which R₂ is as defined for (I), to prepare a compound of formula (IC) in which X represents an —CONH-group;

or with an isothiocyanate of formula R₂—N═C═S (VIIa) in which R₂ is as defined above for (I), to prepare a compound of formula (ID) in which X represents a group —CSNR₂—.

Alternatively, a compound of formula (II) as defined above may be treated with an aryloxycarbonyl halide of formula HalCOOR′₂ in which R′₂ is as defined for (I), to form an intermediate compound of formula: 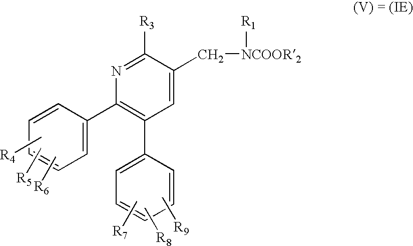
in which the substituents R′₂ and R₁ to R₉ are as defined for (I), which is then treated with an amine of formula R₂R₁₀NH (VI) in which R₂ and R₁₀ are as defined for (I), when a compound of formula (IC) in which X represents a group —CON(R₁₀)— is to be prepared.

The compound of formula (I): (IA), (IB), (IC), (ID) or (IE) thus obtained is optionally converted into an acid-addition salt thereof.

During the preparation of a compound of formula (IA) in which X represents a —CO— group, an activated derivative of the acid of formula (III) may be used, i.e. an acid activated with N,N-dicyclohexylcarbodiimide or with benzotriazol-1-yloxytris(dimethylamino)phosphonium (BOP) hexafluorophosphate, benzotriazol-1-yloxytris(pyrrolidino)phosphonium (PyBOP) hexafluorophosphate or 2-(1H-benzotriazol-1-yl)-1,1,3,3-tetramethyluronium (TBTU) tetrafluoroborate.

During the preparation of a compound of formula (IB) in which X represents an —SO₂— group, the reaction is performed in the presence of a base such as triethylamine or diisopropylethylamine, in a solvent such as dichloromethane or tetrahydrofuran, and at a temperature of between room temperature and the reflex temperature of the solvent.

The compounds of formula (IV) are commercially available or described in the literature, or may be prepared according to methods described therein, such as in J. Org. Chem. USSR, 1970, 6, 2454-2458; J. Am. Chem. Soc., 1952, 74, 2008; J. Med. Chem., 1977, 20(10), 1235-1239; EP 0 469 984; WO 95/18105.

For example, the compounds of formula (IV) may be prepared by halogenation of the corresponding sulfonic acids or of salts thereof, for example the sodium or potassium salts thereof. The reaction is performed in the presence of a halogenating agent such as phosphorous oxychloride, thionylchloride, phosphorous trichloride, phosphorous tribromide or phosphorous pentachloride, without solvent or in a solvent such as a halogenated hydrocarbon or N,N-dimethylformamide and at a temperature of between −10° C. and 200° C.

The aryloxycarbonyl halides that are useful in the preparation of a compound of formula (V) are known or prepared via known methods.

Patent application WO 2002/055 502 describes a compound of formula (II) in which the substituents R₁, R₃, R₆, R₈ and R₉ represent hydrogen and R₄ and R₇ represent a 4-methoxy group.

The compounds of formula: 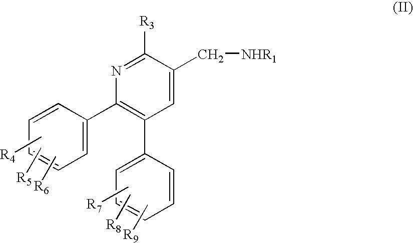
in which:

R₁ represents a hydrogen atom or a (C₁-C₄)alkyl group;

R₃ represents a hydrogen atom or a (C₁-C₄)alkyl, cyano, (C₁-C₄)alkoxymethylene or hydroxymethyl group;

R₄, R₅, R₆, R₇, R₈ and R₉ represent, independently of each other, a hydrogen or halogen atom, a (C₁-C₆)alkyl, (C₁-C₆)alkoxy, trifluoromethyl, trifluoromethoxy, cyano or nitro group or a group S(O)ₙAlk;

are novel on condition that one of the substituents R₁, R₃, R₅, R₆, R₈ and R₉ is other than hydrogen when R₄ and R₇ simultaneously represent a 4-methoxy group.

The compounds of formula (II) are prepared according to the reaction scheme shown below: 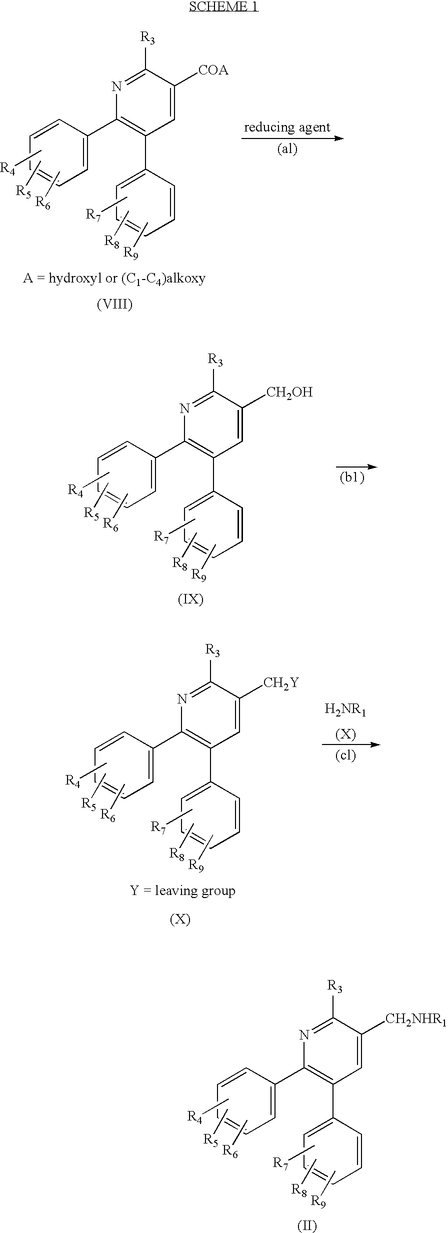
Wherein R₁, R₃ and R₄ to R₉ are as defined for (I).

In step a1), the reaction is performed in the presence of a reducing agent such as sodium borohydride or lithium aluminum hydride, in a solvent such as tetrahydrofuran, and at a temperature of between −20° C. and room temperature. When a compound of formula (VIII) in which A=OH is reduced, the acid may be preactivated by reaction with ethyl chloroformate in the presence of triethylamine.

In step b1), a compound of formula (X) in which Y represents a leaving group as defined above, preferably a halogen atom or an activated hydroxyl group, for instance a methanesulfonate, benzenesulfonate, p-toluenesulfonate or triflate group, is prepared.

Thus, to prepare a compound of formula (X) in which Y represents a halogen atom, a compound of formula (IX) is treated with a halogenating agent such as PCl₅, PBr₃, HBr or BBr₃, in a solvent such as dichloromethane and at a temperature of between 0° C. and room temperature.

To prepare a compound of formula (X) in which Y represents a methanesulfonate, a benzenesulfonate, a p-toluenesulfonate or a trifluoromethanesulfonate, a compound of formula (IX) is reacted with a sulfonyl chloride of formula Z—SO₂—Cl in which Z represents a methyl, a phenyl, a p-tolyl or a trifluoromethyl. The reaction is performed in the presence of a base such as triethylamine, pyridine or N,N-diisopropylethylamine, in a solvent such as dichloromethane or toluene and at a temperature of between −20° C. and the reflux temperature of the solvent.

In step c1), the reaction is performed in a solvent such as N,N-dimethylformamide, acetonitrile, dichloromethane, toluene or 2-propanol, and in the presence or absence of a base. When a base is used, it is chosen from organic bases such as triethylamine, N,N-diisopropylethylamine or N-methylmorpholine. The reaction is performed at a temperature of between 0° C. and the reflux temperature of the solvent.

According to one variant, a compound of formula (II) in which R₁═H may also be prepared by reacting a compound of formula (X) in which Y═Cl with 1,3,5,7-tetraazatricyclo[3.3.1^{3,7}]decane (or hexamethylenetetramine) followed by hydrolysis with a strong acid such as hydrochloric acid.

The compounds of formula (VIII) are prepared according to known methods such as those described in WO 03/082 191 and WO 2005/00817.

Where appropriate, a compound of formula (I) in which Z represents a group N(R₁)XR₂ or N(R₁)COOR′₂ with R₁ other than hydrogen may be prepared by alkylation of a compound of formula (I) in which Z represents a group NHXR₂ or NHCOOR′₂.

According to the present invention, the compounds of formula (IF) in which Z represents a group O—CO—NH—R′₂ may be prepared according to a process characterized in that a compound of formula: 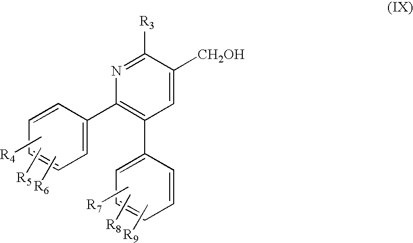
is treated with a compound of formula R′₂—N═C═O.

The compound of formula (IF) thus obtained is optionally converted into an acid-addition salt thereof.

The compounds of formula (VIII) in which R₃ represents a methyl group make it possible to prepare, via methods known to those skilled in the art, the compounds of formula (VIII) in which R₃ represents a hydrogen atom or a (C₂-C₄)alkyl, cyano or (C₁-C₄)alkoxymethyl group.

The compounds of formula (II) in which R₃ represents a (C₁-C₄)alkyl or (C₁-C₄)alkoxymethyl group may also be prepared according to the following reaction scheme:

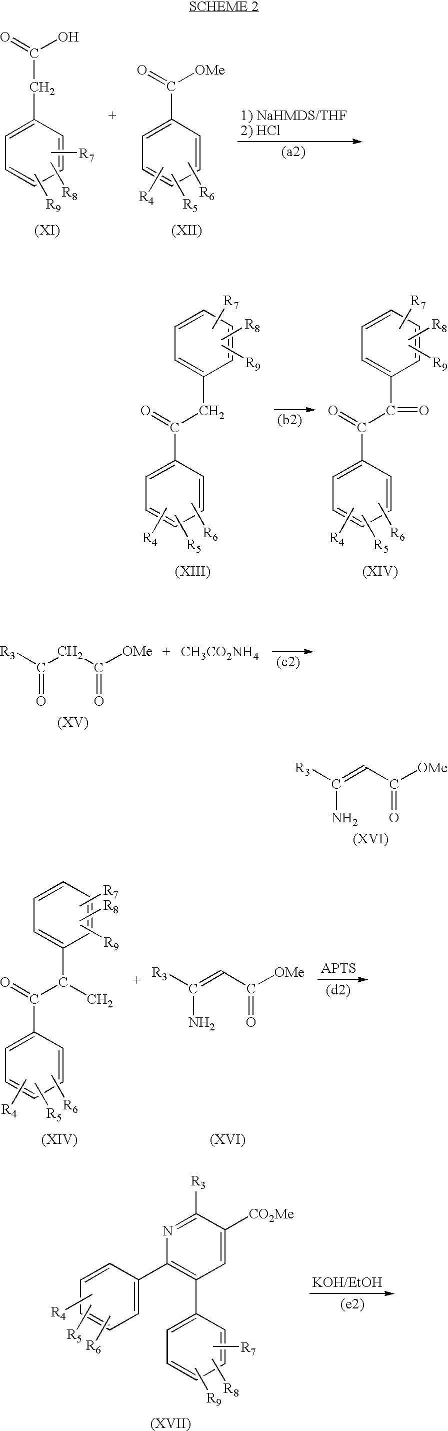

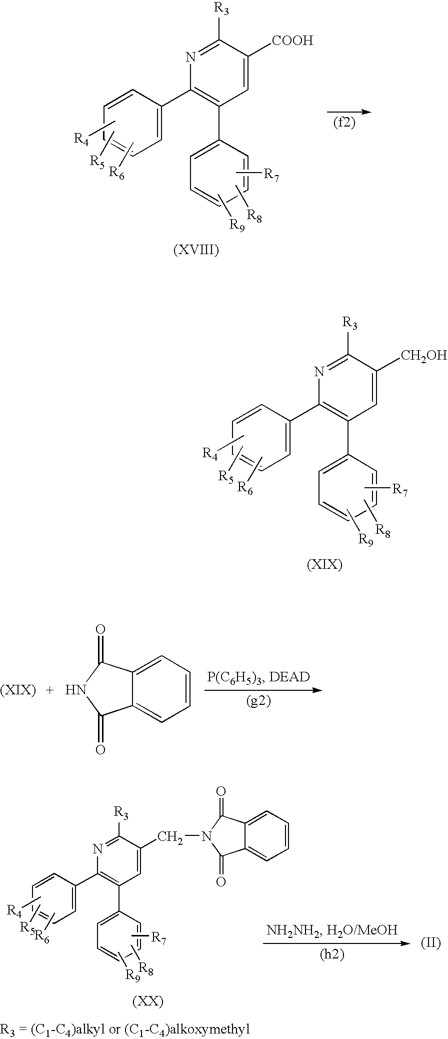

In step (a2), the phenylacetic acid derivative of formula (XI) is treated with the benzoic ester derivative of formula (XII) in the presence of sodium hexamethylenedisilazane (NaHMDS) in a solvent such as THF, and is then acidified to give the compound of formula (XIII); in step (b2), this compound is treated with tetramethylmethanediamine and acetic anhydride to form the compound of formula (XIV).

Moreover, the compound of formula (XVI) is prepared in step (c2) via the action of ammonium acetate on a 3-oxobutanoate derivative of formula (XV). In step (d2), the nicotinic ester of formula (XVII) is then prepared via the action of compound (XIV) on compound (XVI) in the presence of para-toluenesulfonic acid (PTSA).

This ester is hydrolyzed in basic medium in step (e2) and the acid function is then reduced in step (f2), for example with the borane/THF complex.

Alternatively, to perform step (f2), an anhydride may be prepared as an intermediate, and then reduced, for example via the action of a metal borohydride. The ester of formula (XVII) may also be directly reduced with reducing agents to the alcohol of formula (XIX).

In step (g2), the compound of formula (XIX) bearing a hydroxymethyl group is employed in a Mitsunobu reaction in the presence of phthalimide to give a compound of formula (XX), which, when treated with hydrazine hydrate, in a final step (h2), gives the expected compound (II).

The compounds of formula (I) in which R₃═CH₂OH may be prepared from the compounds of formula (I) in which R₃═CH₂OMe via a demethylation reaction, for example in the presence of a Lewis acid such as BBr₃.

The EXAMPLES that follow describe the preparation of certain compounds in accordance with the invention. These examples are not limiting and merely illustrate the present invention. The numbers of the illustrated compounds refer to those given in TABLES I, II and III below.

In the Preparations and in the Examples, the following abbreviations are used:
ether: diethyl ether
iso ether: diisopropyl ether
DMSO: dimethyl sulfoxide
DIPEA: diisopropylethylamine
DMF: N,N-dimethylformamide
THF: tetrahydrofuran
DCM: dichloromethane
EtOAc: ethyl acetate
PyBOP: benzotriazol-1-yloxytris(pyrrolidino)phosphonium hexafluorophosphate
TBTU: 2-(1H-benzotriazol-1-yl)oxytris(pyrrolidino)phosphonium tetrafluoroborate
NaHMDS: sodium hexamethylenedisilazane
PTSA: para-toluene sulfonic acid
2N hydrochloric ether: 2N solution of hydrogen chloride in diethyl ether
DEAD: diethyl azodicarboxylate
pH2 buffer solution: solution of 16.66 g of KHSO₄ and 32.32 g of K₂SO₄ in 1 liter of water.
m.p.: melting point
RT: room temperature

The nuclear magnetic resonance spectra are recorded at 200 MHz in DMSO-d₆. For the interpretation of the spectra, the following abbreviations are used: s: singlet, d: doublet, t: triplet, unres. comp.: unresolved complex, mt: multiplet, bs: broad singlet.

The compounds according to the invention are analyzed by LC/UV/MS coupling (liquid chromatography/UV detection/mass spectrometry). The molecular peak (MH⁺) and the retention time (t) in minutes are measured.

Conditions A:

An Xterra Waters®0 MS C18 column, sold by Waters, of 2.1×30 mm, 3.5 μm, is used at room temperature, with a flow rate of 1 ml/minute.

The eluent is composed as follows:

solvent A: 0.025% of trifluoroacetic acid (TFA) in water

solvent B: 0.025% of TFA in acetonitrile.

Gradient: the percentage of solvent B ranges from 0 to 100% over 2 minutes with a steady stage at 100% of B for 1 minute.

The UV detection is performed at between 210 nm and 400 nm and the mass detection in chemical ionization mode is performed at atmospheric pressure.

Conditions: MS2

An XTerra MS C18 column of 2.1×30 mm, 3.5 μm is used, at 30° C. and with a flow rate of 0.8 ml/minute.

The eluent is composed as follows:

solvent A: 0.025% of trifluoroacetic acid (TFA) in water;

solvent B: 0.025% of TFA in acetonitrile.

Gradient:

| Time (minutes) | % A | % B |
|---|---|---|
| 0 | 100 | 0 |
| 2 | 0 | 100 |
| 2.7 | 0 | 100 |
| 2.75 | 100 | 0 |

The UV detection is performed using a diode array detector at between 210 and 400 nm and the mass detection is performed in positive ESI chemical ionization mode.

Conditions MS5

An XTerra MS C18 column of 2.1×30 mm, 3.5 μm is used, at a flow rate of 1 ml/minute.

The solvent is composed as follows:
Solvent A: 0.025% of TFA in water,
Solvent B: 0.025% of TFA in acetonitrile.

Gradient

| Time (minutes) | % A | % B |
|---|---|---|
| 0 | 100 | 0 |
| 2 | 0 | 100 |
| 2.7 | 0 | 100 |
| 2.75 | 100 | 0 |

The UV detection is performed with a diode array detector at between 210 and 400 nm and the mass detection is performed in positive ESI mode.

Preparation 1

### 1-(6-(4-chlorophenyl)-5-(2,4-dichlorophenyl)-2-methylpyridin-3-y)methanamine

### A) 1-(4-chlorophenyl)-2-(2,4-dichlorophenyl)prop-2-en-1-one

10 g of 1-(4-chlorophenyl)-2-(2,4-dichlorophenyl)ethanone, 17 ml of N,N,N,N-tetramethylmethanediamine and 17 ml of acetic anhydride are mixed together at RT; the mixture is heated at 90° C. for 3 hours and then allowed to cool to RT. The mixture is poured into crushed ice and then filtered. The solid is dried under vacuum. 10 g of the expected compound are obtained, m.p.=89° C.

### B) ethyl 5-(2,4-dichlorophenyl)-6-(4-chlorophenyl)-2-methylpyridine-3-carboxylate

A mixture containing 7 g of the compound from the preceding step, 2.62 g of ethyl 3-aminobute-2-enoate and 140 mg of para-toluenesulfonic acid is prepared in 60 ml of n-butanol and then heated for 24 hours at the reflux temperature of the solvent. Three quarters of the solvent is evaporated off and 80 ml of pentane at 0° C. are then added. The precipitate formed is filtered off and the filtrate is concentrated. The residue is chromatographed on silica, eluting with a cyclohexane/EtOAc mixture (90/10; v/v). 7 g of the expected compound are obtained, m.p.=114° C.

### C) (6-(4-chlorophenyl)-5-(2,4-dichlorophenyl)-2-methylpyridin-3-yl)methanol

8.4 g of the compound obtained in the preceding step are placed in 200 ml of THF, 0.75 g of LiAlH₄ is added slowly at RT and the mixture is stirred for 1 hour at RT. 100 ml of ether, 1 ml of water, 1 ml of 4N sodium hydroxide and 3 ml of water are added. The salts formed are filtered off and the product is then crystallized from a minimum amount of DCM and filtered off. 7 g of the expected compound are obtained.

### D) 3-chloromethyl-6-(4-chlorophenyl)-5-(2,4-dichlorophenyl)-2-methylpyridine

7 g of the compound obtained in the preceding step are placed in 150 ml of DCM under nitrogen, and 4 g of PCl₅ are added slowly, at 0° C. The mixture is stirred for 1 hour at RT and then washed with water and extracted with DCM. The extracts are dried, filtered and evaporated to give 7.2 g of the expected compound.

### E) 1-(6-(4-chlorophenyl)-5-(2,4-dichlorophenyl)-2-methylpyridin-3-yl)methanamine hydrochloride

7.2 g of the compound obtained in the preceding step are placed in 200 ml of ethanol under nitrogen with 3.05 g of hexamethylenetetramine and 2.7 g of Nal, and the mixture is stirred at RT for 16 hours. 20 ml of concentrated HCl are added and the mixture is then refluxed for 1 hour. The precipitate formed is filtered off. The filtrate is evaporated to dryness and then taken up in 100 ml of water. The impurities are extracted with EtOAc. The aqueous phase is basified with 8% NaOH and the product is extracted with EtOAc. The organic phase is dried over MgSO₄, evaporated to dryness, taken up in Et₂O and treated with HCl/Et₂O. The white precipitate obtained is filtered off and dried under vacuum. 5 g of the expected compound are obtained.

MH⁺=377; t=6.85 mn.

¹H NMR: 2.65 ppm: s: 3H; 4.20 ppm: q: 2H; 7.10 to 8.00 ppm: m: 8H; 8.40 ppm: bs: 3H.

Preparation 2

### 1-(6-(4-bromophenyl)-5-(2,4-dichlorophenyl)-2-methylpyridin-3-yl)methanamine hydrochloride

This compound is prepared according to the procedure of Preparation 1.

MH⁺=421; t=6.05 mn.

Preparation 3

### 1-(6-(4-methoxyphenyl)-5-(2,4-dichlorophenyl)-2-methylpyridin-3-yl)methanamine hydrochloride

MH⁺=373.0; t=6.37.

Preparation 4

### 1-(5-(2,4-dichlorophenyl)-2-(methoxymethyl)-6-(4-methoxyphenyl)pyridin-3-yl)methanamine

### A) 2-(2,4-dichlorophenyl)-1-(4-methoxyphenyl)ethanone

150 ml of NaHMDS at −78° C. diluted with 150 ml of THF are placed under nitrogen and 25 g of 2,4-dichlorophenylacetic acid dissolved in 150 ml of THF are added dropwise. At −78° C., after stirring for 2 hours, 20.26 g of methyl 4-methoxybenzoate dissolved in 150 ml of TBF are added. The mixture is allowed to warm to 0° C. and stirring is continued for 2 hours at this temperature. At 0° C., 10% hydrochloric acid is added dropwise in an amount sufficient to hydrolyze the reaction medium, and the mixture is stirred for 2 hours at RT. The resulting mixture is extracted with ether and the organic phase is then dried over Na₂SO₄. The solid formed is taken up in 250 ml of pentane and then stirred, filtered and oven-dried. 23.43 g of the expected compound are obtained.

LC/MS (conditions A). MH⁺⁼295.0; t=10.34.

### B) 2-(2,4-dichlorophenyl)-1-(4-methoxyphenyl)prop-2-en-1-one

A mixture containing 23 g of the compound obtained in the preceding step and 40.21 g of tetramethylmethanediamine is prepared, to which are added dropwise 24 g of acetic anhydride, and the mixture is then stirred for 2 hours at 90° C. After cooling to RT, 500 ml of water are added. The precipitate formed is filtered off and then rinsed several times with water. The solid obtained is dried under vacuum. 22.93 g are obtained and are used in unmodified form in the following step.

LC/MS (conditions A). MH⁺=307.0; t10.47.

### C) methyl 3-amino-4-methoxybut-2-enoate

A mixture containing 30 g of presublimed ammonium acetate, 165 ml of cyclohexane and 15 ml of methyl 4-methoxy-3-oxobutanoate and 4 Å molecular sieves is placed under nitrogen. The mixture is stirred for 4 hours at 90° C. and then evaporated to dryness. The residue is washed and extracted with DCM and the organic phase is then filtered and evaporated. The product obtained is used without further purification in the following step.

### D) 5-(2,4-dichlorophenyl)-2-(methoxymethyl)-6-(4-methoxyphenyl)nicotinate

A mixture containing 10.63 g of the compound obtained in step C, 22.5 g of the compound obtained in step B, 0.50 g of PTSA and 54 ml of butanol is prepared. It is stirred at 150° C. for 3 hours. The butanol is evaporated off and the residue is then taken up in 400 ml of DCM. The organic phase is washed with 400 ml of water and then dried over Na₂SO₄, filtered and evaporated to dryness. 25.14 g of the expected compound are obtained in crude form.

### E) 5-(2,4-dichlorophenyl)-2-(methoxymethyl)-6-(4-methoxyphenyl)nicotinic acid

25.1 g of the compound obtained in the preceding step are placed in 232 ml of ethanol in the presence of 32.5 g of potassium hydroxide, and the mixture is refluxed for 3 hours. After cooling to RT, the solvent is evaporated off and the residue is then treated with 300 ml of water. The aqueous phase is acidified with concentrated HCl and then extracted with 300 ml of ether. The organic phase is dried over Na₂SO₄, filtered and evaporated to dryness. The product obtained is recrystallized from an ether/pentane mixture to give 15.11 g of the expected compound.

### F) (5-(2,4-dichlorophenyl)-2-(methoxymethyl)-6-(4-methoxyphenyl)pyridin-3-yl)methanol

A mixture containing 80 ml of 1M BH₃ in THF is prepared under nitrogen and, at 0° C., 13.4 g of the compound obtained in the preceding step in 200 ml of THF are added dropwise. The mixture is stirred overnight at RT, followed by dropwise addition of 125 ml of methanol and then, at 0° C., 250 ml of hydrochloric ether, and the mixture is stirred for 3 hours. The ether phase is dried and then taken up in 250 ml of ether and washed with saturated NaHCO₃ solution, and then with water. The organic phase is then dried over Na₂SO₄, filtered and evaporated to dryness. The crude product obtained is chromatographed on silica, eluting with CH₂Cl₂ containing from 0 to 5% MeOH. 1.40 g of the expected compound are obtained.

LC/MS (conditions A). MH⁺=404.0; t=9.06.

### G) 2-((5-2-(2,4-dichlorophenyl)-2-(methoxymethyl)-6-(4-methoxyphenyl)pyridin-3-yl)methyl)-1H-isoindole-1,3-(2H)dione

A mixture is prepared containing 1.4 g of the compound obtained in the preceding step, 0.9 g of triphenylphosphine, 0.51 g of phthalimide and 0.6 ml of ether, and 0.62 g of DEAD is added dropwise thereto at −10° C. After leaving overnight at RT, the reaction medium is diluted with 100 ml of ether and then washed with 100 ml of pH2 buffer, 100 ml of saturated NaHCO₃ solution and 100 ml of saturated NaCl solution, and the organic phase is then dried over Na₂SO₄. The crude product obtained is purified by chromatography on silica, eluting with a DCM/MeOH mixture (97/3; v/v). 1.8 g of the expected compound are obtained.

LC/MS (conditions A). MH⁺=533.0; t=9.79.

### H) 1-(5-(2,4-dichlorophenyl)-2-(methoxyphenyl)-6-(4-methoxyphenyl)pyridin-3-yl)methanamine

A mixture containing 1.79 g of the compound obtained previously and 0.31 ml of hydrazine hydrate in 45 ml of methanol is placed under nitrogen and refluxed for 3 hours. 100 ml of water and 100 ml of DCM are added and the organic phase is then washed with 100 ml of 10% NaOH solution, 100 ml of saturated NaHCO₃ solution and 100 ml of saturated NaCl solution. The organic phase is dried over Na₂SO₄ and evaporated to dryness. 0.944 g of the expected compound is obtained.

LC/MS (conditions A). MH⁺=403.0; t=6.58.

Preparation 5

### 1-(6-(4-chlorophenyl)-5-(2,4-dichlorophenyl)-2-(methoxymethyl)pyridin-3-yl)methanamine

This compound is prepared according to the procedure described in Preparation 4.

LC/MS (conditions A). MH⁺=407.0, t=7.15.

### EXAMPLE 1

### Compound 3

### N-{[-6-(4-chlorophenyl)-5-(2,4-dichlorophenyl)-2-methylpyridin-3-yl]methyl}-1H-indole-2-carboxamide

0.5 g of the compound of Preparation 1, 0.19 g of 2-indolecarboxylic acid, 0.75 g of PyBOP and 0.34 ml of triethylamine are placed in 10 ml of DCM and stirred for 2 hours at RT. The reaction medium is washed with 3% HCl, with water, with aqueous 8% sodium hydroxide solution and with water. The resulting mixture is extracted with DCM, dried, filtered and evaporated. The product is chromatographed on silica, eluting with a DCM/MeOH mixture with a gradient of from 100/0 to 95/5, to give 130 mg of the expected compound.

¹H NMR: 2.65 ppm: s: 3H; 4.60 ppm: d: 2H; 6.90 to 7.70 ppm: unres. comp.: 13H; 9.05 ppm: t: 1H; 11.62 ppm: bs: 1H.

### EXAMPLE 2

### Compound 52

### N-{[-6-(4-chlorophenyl)-5-(2,4-dichlorophenyl)-2-methylpyridin-3-yl]methyl}-4-(trifluoromethyl)benzenesulfonamide

0.5 g of the compound of Preparation 1, 0.29 g of 4-trifluoromethylbenzenesulfonyl chloride and 0.34 ml of triethylamine are placed in 10 ml of DCM and stirred for 2 hours at RT. The reaction medium is washed with 3% HCl, with water, with aqueous 8% sodium hydroxide solution and with water. The mixture is extracted with DCM, dried, filtered and evaporated. The residue is chromatographed on silica, eluting with a DCM/MeOH mixture with a gradient of from 100/0 to 95/5, to give 400 mg of the expected compound.

¹H NMR: 2.51 ppm: s: 3H; 4.20 ppm: s: 2H; 7.10 to 7.70 ppm: unres. comp.: 8H; 7.80 to 8.10 ppm: 2d: 4H; 8.50 ppm: s: 1H.

### EXAMPLE 3

### Compound 81

### N-{[-6-(4-chlorophenyl)-5-(2,4-dichlorophenyl)-2-methylpyridin-3-yl]methyl}-N′-[4-(trifluoromethyl)phenyl]urea

0.5 g of the compound of Preparation 1 and 0.22 g of 1-isocyanato-4-(trifluoromethyl)benzene are placed in 10 ml of DCM and stirred for 2 hours at RT. The reaction medium is washed with 3% HCl, with water, with aqueous 8% sodium hydroxide solution and with water. The mixture is extracted with DCM, dried, filtered and evaporated. The residue is chromatographed on silica, eluting with a DCM/MeOH mixture with a gradient of from 100/0 to 95/5, to give 400 mg of the expected compound.

¹H NMR: 2.60 ppm: s: 3H; 4.40 ppm: d: 2H; 6.87 ppm: t: 1H; 7.20 to 7.65 ppm: unres. comp.: 12H; 9.03 ppm: s: 1H.

### EXAMPLE 4

### Compound 121 [6-(4-chlorophenyl)-5-(2,4-dichlorophenyl)-2-methylpiperidin-3-yl]methylphenylcarbamate

0.5 g of the compound of Preparation 1, step C and 0.28 ml of benzene isocyanate are placed in 18 ml of DCM and stirred at reflux for 1 hour. After cooling to RT, the reaction medium is treated with 200 ml of water and 150 ml of DCM. The phases are separated by settling. The organic phase is washed with 200 ml of water, dried over Na₂SO₄ and evaporated to dryness. The product is purified on a column of silica, eluting with a DCM/MeOH mixture (100/0 to 97/3; v/v). 334.8 mg of the expected compound are obtained.

### EXAMPLE 5

The compounds of formula (IA) 15 to 50 and (IB) 60 to 88 in which Z represents N(R₁)XR₂ and —X—═—CO— or SO₂ are prepared by combinatorial chemistry according to the process described below:

A carboxylic acid of formula (III) or, respectively, a sulfonyl halide of formula (IV) is dissolved in DMF to a concentration of 0.25M in the presence of 3 equivalents of DIPEA. 120 μl of this solution and 120 μl of a solution of TBTU in DMF at a concentration of 0.25M are placed in each 2 ml well. 300 μl of a solution containing the corresponding compound of formula (II) in DMF at a concentration of 0.1M and 3 equivalents of DIPEA is added to each well. The plates are shaken at RT for 16 hours and then evaporated. The products formed are dissolved in each well with 500 μl of EtOAc, 400 μl of 0.1M Na₂CO₃ are added and the plates are shaken. After separation of the phases by settling, 430 μl of aqueous phase are discharged and 300 μl of 5% NaCl are then added and the plates are shaken. 350 μl of aqueous phase are then discarded and the residues are analyzed by LC/UV/MS.

### EXAMPLE 6

The compounds of formula (IC) 87 to 116 and (ID) 117 to 119 in which Z represents N(R₁)XR₂ and —X—═—CON(R₁₀) or —CSN(R₁₀) are prepared by combinatorial chemistry according to the process described below:

An isocyanate of formula (VII) or, respectively, thioisocyanate of formula (VIIa) is dissolved in THF to a concentration of 0.25M in the presence of 3 equivalents of DIPEA. 120 μl of this solution and 120 μl of a solution of TBTU in DMF at a concentration of 0.25M are placed in each 2 ml well. 300 μl of a solution containing the corresponding compound of formula (II) in DMF at a concentration of 0.1M and 3 equivalents of DIPEA are added to each well. The plates are shaken at RT for 16 hours and then evaporated. The products formed are dissolved in each well with 500 μl of EtOAc, 400 μl of 0.1M Na₂CO₃ are added and the plates are shaken. After separation of the phases by settling, 430 μl of aqueous phase are discarded and 300 μl of 5% NaCl are then added and the plates are shaken. 350 μl of aqueous phase are then discarded and the residues are analyzed by LC/UV/MS.

The tables that follow illustrate the chemical structures and the physical properties of a number of compounds according to the invention.

In these tables, Me, Et, Pr, nBu and tBu represent methyl, ethyl, propyl, n-butyl and tert-butyl groups, respectively.

The conditions used for the LC/MS analysis of the compounds are indicated by A, MS5 or MS2.

TABLE 1

| | |
|---|---|
| | (IA) |
| 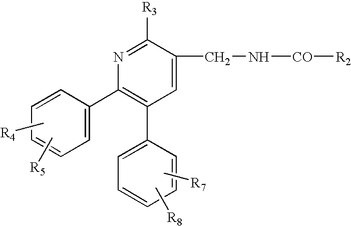 | |

| Compound | | | | | |
|---|---|---|---|---|---|
| No. | R₂ | R₃ | R₄, R₅ | R₇, R₈ | Characterization |
| 1 | 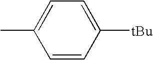 | Me | 4-Cl | 2,4-diCl | MH⁺ = 537 t = 11.58 A m.p. = 202° C. |
| 2 | 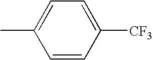 | Me | 4-Cl | 2,4-diCl | MH⁺ = 549 t = 11.20 A m.p. = 101.5° C. |
| 3 | 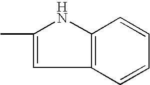 | Me | 4-Cl | 2,4-diCl | MH⁺ = 520 t = 10.60 A m.p. = 135.5° C. |
| 4 | 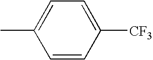 | Me | 4-Br | 2,4-diCl | MH⁺ = 593 t = 11.54 A m.p. = 102° C. |
| 5 | 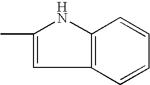 | Me | 4-Br | 2,4-diCl | MH⁺ = 564 t = 10.84 A m.p. = 126° C. |
| 6 | 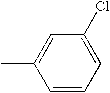 | Me | 4-Cl | 2,4-diCl | MH⁺ = 515.0 t = 11.31 A |
| 7 | 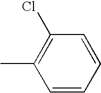 | Me | 4-Cl | 2,4-diCl | MH⁺ = 515.0 t = 10.91 A |
| 8 | 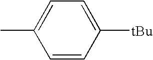 | CH₂OMe | 4-Cl | 2,4-diCl | MH⁺ = 567.0 t = 12.44 A |
| 9 | 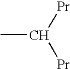 | CH₂OMe | 4-OMe | 2,4-diCl | MH⁺ = 529.0 t = 11.10 A |
| 10 | 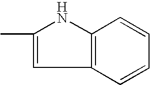 | CH₂OMe | 4-OMe | 2,4-diCl | MH⁺ = 546.2 t = 10.45 A |
| 11 | 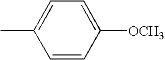 | CH₂OMe | 4-Cl | 2,4-diCl | MH⁺ = 595.2 t = 11.99 A |
| 12 | 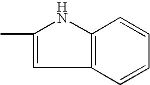 | CH₂OMe | 4-Cl | 2 4-diCl | MH⁺ = 550.0 t = 11.38 A |
| 13 | 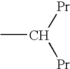 | CH₂OMe | 4-Cl | 2,4-diCl | MH⁺ = 533.0 t = 12.07 A |
| 14 | 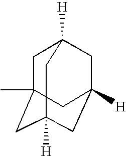 | CH₂OMe | 4-Cl | 2,4-diCl | MH⁺ = 569.0 t = 12.52 A |
| 15 | 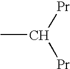 | Me | 4-OMe | 2,4-diCl | MH⁺ = 470.9 t = 1.65 MS5 |
| 16 | 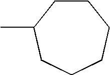 | Me | 4-OMe | 2,4-diCl | MH⁺ = 496.9 t = 1.70 MS5 |
| 17 | 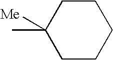 | Me | 4-OMe | 2,4-diCl | MH⁺ = 496.9 t = 1.65 MS5 |
| 18 | 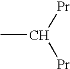 | Me | 4-OMe | 2,4-diCl | MH⁺ = 499.0 t = 1.71 MS5 |
| 19 | 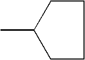 | Me | 4-OMe | 2,4-diCl | MH⁺ = 469.0 t = 1.63 MS5 |
| 20 | —Bu | Me | 4-OMe | 2,4-diCl | MH⁺ = 456.9 |
| | | | | | t = 1.63 |
| | | | | | MS5 |
| 21 | 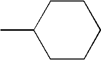 | Me | 4-OMe | 2,4-diCl | MH⁺ = 483.0 t = 1.67 MS5 |
| 22 | 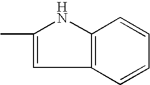 | Me | 4-OMe | 2,4-diCl | MH⁺ = 515.9 t = 1.78 MS5 |
| 23 | 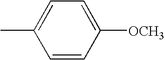 | Me | 4-OMe | 2,4-diCl | MH⁺ = 560.9 t = 1.74 MS5 |
| 24 | 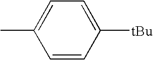 | Me | 4-OMe | 2,4-diCl | MH⁺ = 533.0 t = 1.79 MS5 |
| 25 | 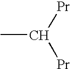 | Me | 4-Cl | 2,4-diCl | MH⁺ = 474.9 t = 1.74 MS5 |
| 26 | 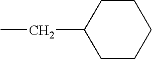 | Me | 4-Cl | 2,4-diCl | MH⁺ = 500.9 t = 1.84 MS5 |
| 27 | 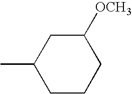 | Me | 4-Cl | 2,4-diCl | MH⁺ = 516.9 t = 1.70 MS5 |
| 28 | 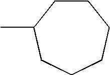 | Me | 4-Cl | 2,4-diCl | MH⁺ = 500.9 t = 1.81 MS5 |
| 29 | 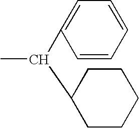 | Me | 4-Cl | 2,4-diCl | MH⁺ = 576.9 t = 2.03 MS5 |
| 30 | 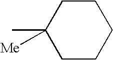 | Me | 4-Cl | 2,4-diCl | MH⁺ = 500.9 t = 1.81 MS5 |
| 31 | 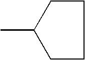 | Me | 4-Cl | 2,4-diCl | MH⁺ = 472.9 t = 1.74 MS5 |
| 32 | 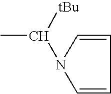 | Me | 4-Cl | 2,4-diCl | MH⁺ = 539.9 t = 1.99 MS5 |
| 33 | 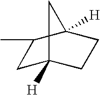 | Me | 4-Cl | 2,4-diCl | MH⁺ = 498.9 t = 1.84 MS5 |
| 34 | 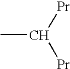 | Me | 4-Cl | 2,4-diCl | MH⁺ = 502.9 t = 1.90 MS5 |
| 35 | 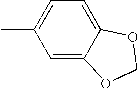 | Me | 4-Cl | 2,4-diCl | MH⁺ = 524.8 t = 1.72 MS5 |
| 36 | —Bu | Me | 4-Cl | 2,4-diCl | MH⁺ = 460.9 |
| | | | | | t = 1.76 |
| | | | | | MS5 |
| 37 | 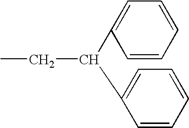 | Me | 4-Cl | 2,4-diCl | MH⁺ = 584.8 t = 1.84 MS5 |
| 38 | 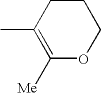 | Me | 4-Cl | 2,4-diCl | MH⁺ = 500.9 t = 1.67 MS5 |
| 39 | 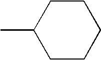 | Me | 4-Cl | 2,4-diCl | MH⁺ = 486.9 t = 1.77 MS5 |
| 40 | 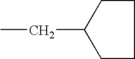 | Me | 4-Cl | 2,4-diCl | MH⁺ = 486.9 t = 1.81 MS5 |
| 41 | 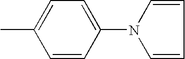 | Me | 4-Cl | 2,4-diCl | MH⁺ = 545.8 t = 1.87 MS5 |
| 42 | 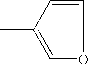 | Me | 4-Cl | 2,4-diCl | MH⁺ = 470.8 t = 1.67 MS5 |
| 43 | 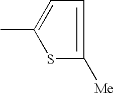 | Me | 4-Cl | 2,4-diCl | MH⁺ = 500.8 t = 1.76 MS5 |
| 44 | 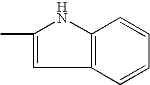 | Me | 4-Cl | 2,4-diCl | MH⁺ = 519.8 t = 1.83 MS5 |
| 45 | 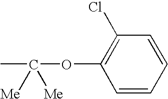 | Me | 4-Cl | 2,4-diCl | MH⁺ = 572.8 t = 1.99 MS5 |
| 46 | 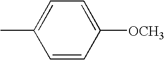 | Me | 4-Cl | 2,4-diCl | MH⁺ = 564.8 t = 1.87 MS5 |
| 47 | 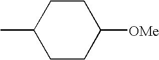 | Me | 4-Cl | 2,4-diCl | MH⁺ = 516.9 t = 1.69 MS5 |
| 48 | 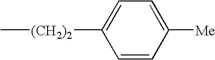 | Me | 4-Cl | 2,4-diCl | MH⁺ = 522.8 MS5 |
| 49 | 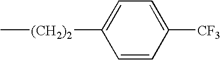 | Me | 4-Cl | 2,4-diCl | MH⁺ = 576.8 t = 1.85 MS5 |
| 50 | 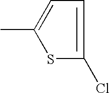 | Me | 4-Cl | 2,4-diCl | MH⁺ = 520.7 t = 1.84 MS5 |

TABLE 2

| | |
|---|---|
| | (IB) |
| 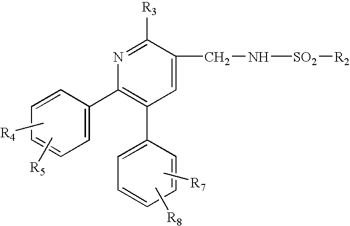 | |

| Compound | | | | | |
|---|---|---|---|---|---|
| No. | R₂ | R₃ | R₄, R₅ | R₇, R₈ | Characterization |
| 51 | 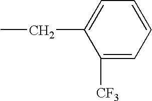 | Me | 4-Cl | 2,4-diCl | MH⁺ = 599 t = 11.45 A m.p. = 86° C. |
| 52 | 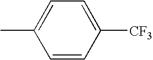 | Me | 4-Cl | 2,4-diCl | MH⁺ = 585 t = 11.66 A m.p. = 98° C. |
| 53 | 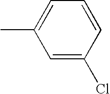 | Me | 4-Cl | 2,4-diCl | MH⁺ = 551 t = 11.47 A m.p. = 94° C. |
| 54 | —nBu | Me | 4-Cl | 2,4-diCl | MH⁺ = 498 |
| | | | | | t = 10.92 |
| | | | | | A |
| | | | | | m.p. 66° C. |
| 55 | 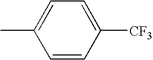 | Me | 4-Br | 2,4-diCl | MH⁺ = 629 t = 11.62 A m.p. = 207° C. |
| 56 | 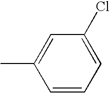 | CH₂OMe | 4-Cl | 2,4-diCl | MH⁺ = 581.0 t = 11.88 A |
| 57 | 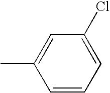 | CH₂OMe | 4-OMe | 2,4-diCl | MH⁺ = 576.8 t = 11.01 A |
| 59 | 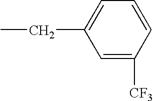 | CH₂OMe | 4-Cl | 2,4-diCl | MH⁺ 628.8 t = 11.92 A |
| 60 | 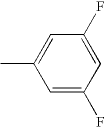 | Me | 4-OMe | 2,4-diCl | MH⁺ = 549.5 t = 1.68 MS2 |
| 61 | 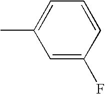 | Me | 4-OMe | 2,4-diCl | MH⁺ = 531.5 t = 1.64 MS2 |
| 62 | 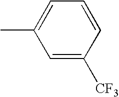 | Me | 4-OMe | 2,4-diCl | MH⁺ = 581.5 t = 1.72 MS2 |
| 63 | 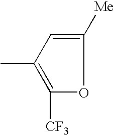 | Me | 4-Cl | 2,4-diCl | MH⁺ = 588.7 t = 2.04 MS5 |
| 64 | 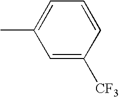 | Me | 4-Cl | 2,4-diCl | MH⁺ = 584.7 t = 2.02 MS5 |
| 65 | 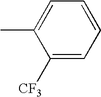 | Me | 4-Cl | 2,4-diCl | MH⁺ = 584.7 t = 1.97 MS5 |
| 66 | 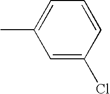 | Me | 4-Cl | 2,4-diCl | MH⁺ = 550.8 t = 1.98 MS5 |
| 67 | 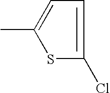 | Me | 4-Cl | 2,4-diCl | MH⁺ = 556.7 t = 2.01 MS5 |
| 68 | 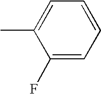 | Me | 4-Cl | 2,4-diCl | MH⁺ = 534.8 t = 1.86 MS5 |
| 69 | 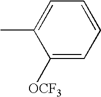 | Me | 4-Cl | 2,4-diCl | MH⁺ = 600.7 t = 2.01 MS5 |
| 70 | 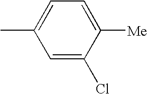 | Me | 4-Cl | 2,4-diCl | MH⁺ = 564.8 t = 2.04 MS5 |
| 71 | 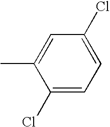 | Me | 4-Cl | 2,4-diCl | MH⁺ = 585.4 t = 1.97 MS2 |
| 72 | 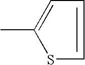 | Me | 4-Cl | 2,4-diCl | MH⁺ = 523.4 t = 1.82 MS2 |
| 73 | 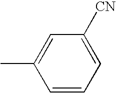 | Me | 4-Cl | 2,4-diCl | MH⁺ = 542.5 t = 1.89 MS2 |
| 74 | 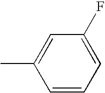 | Me | 4-CL | 2,4-diCl | MH⁺ = 535.5 t = 1.85 MS2 |
| 75 | —Bu | Me | 4-Cl | 2,4-diCl | MH⁺ = 497.5 |
| | | | | | t = 1.80 |
| | | | | | MS2 |
| 76 | 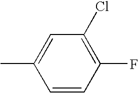 | Me | 4-Cl | 2,4-diCl | MH⁺ = 569.4 t = 1.93 MS2 |
| 77 | 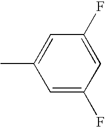 | Me | 4-Cl | 2,4-diCl | MH⁺ = 553.5 t = 1.89 MS2 |

TABLE 3

| | |
|---|---|
| | (IC) |
| 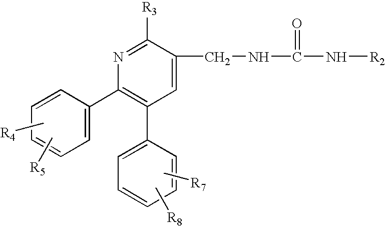 | |

| Compound | | | | | |
|---|---|---|---|---|---|
| No. | R₂ | R₃ | R₄, R₅ | R₇, R₈ | Characterization |
| 78 | 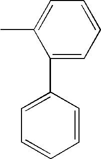 | Me | 4-Cl | 2,4-diCl | MH⁺ = 572 t = 10.96 A m.p. = 123.5° C. |
| 79 | 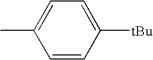 | Me | 4-Cl | 2,4-diCl | MH⁺ = 552 t = 11.28 A m.p. = 208° C. |
| 80 | 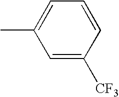 | Me | 4-Cl | 2,4-diCl | MH⁺ = 564 t = 11.06 A m.p. = 110° C. |
| 81 | 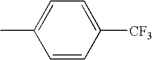 | Me | 4-Cl | 2,4-diCl | MH⁺ = 564 t = 11.14 A — |
| 82 | 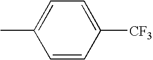 | Me | 4-Br | 2,4-diCl | MH⁺ = 608 t = 11.50 A m.p. = 230° C. |
| 83 | 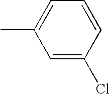 | Me | 4-Cl | 2,4-diCl | MH⁺ = 530 t = 11.20 A |
| 84 | 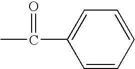 | Me | 4-Cl | 2,4-diCl | MH⁺ = 524.0 t = 10.98 A |
| 85 | 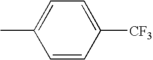 | CH₂OMe | 4-Cl | 2,4-diCl | MH⁺ = 594.0 t = 11.91 A |
| 86 | 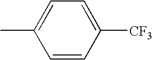 | CH₂OMe | 4-OMe | 2,4-diCl | MH⁺ = 590.0 t = 10.96 A |
| 87 | 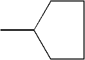 | Me | 4-OMe | 2,4-diCl | MH⁺ = 484.6 t = 1.49 MS2 |
| 88 | 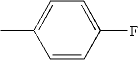 | Me | 4-OMe | 2,4-diCl | MH⁺ = 510.6 t = 1.55 MS2 |
| 89 | 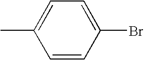 | Me | 4-OMe | 2,4-diCl | MH⁺ = 570.5 t = 1.65 MS2 |
| 90 | 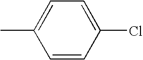 | Me | 4-OMe | 2,4-diCl | MH⁺ = 526.5 t = 1.61 MS2 |
| 91 | 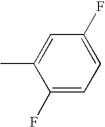 | Me | 4-OMe | 2,4-diCl | MH⁺ = 528.6 t = 1.59 MS2 |
| 92 | —tBu | Me | 4-OMe | 2,4-diCl | MH⁺ = 472.6 |
| | | | | | t = 1.49 |
| | | | | | MS2 |
| 93 | 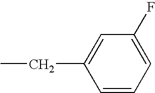 | Me | 4-OMe | 2,4-diCl | MH⁺ = 524.6 t = 1.51 MS2 |
| 94 | 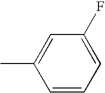 | Me | 4-OMe | 2,4-diCl | MH⁺ = 510.6 t = 1.57 MS2 |
| 95 | 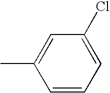 | Me | 4-OMe | 2,4-diCl | MH⁺ = 526.5 t = 1.62 MS2 |
| 96 | 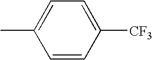 | Me | 4-OMe | 2,4-diCl | MH⁺ = 560.6 t = 1.67 MS2 |
| 97 | 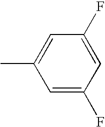 | Me | 4-OMe | 2,4-diCl | MH⁺ = 528.6 t = 1.63 MS2 |
| 98 | 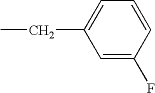 | Me | 4-Cl | 2,4-diCl | MH⁺ = 527.8 t = 1.70 MS5 |
| 99 | —tBu | Me | 4-Cl | 2,4-diCl | MH⁺ = 475.9 |
| | | | | | t = 1.68 |
| | | | | | MS5 |
| 100 | 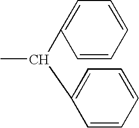 | Me | 4-Cl | 2,4-diCl | MH⁺ = 585.8 t = 1.83 MS5 |
| 101 | 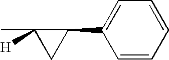 | Me | 4-Cl | 2,4-diCl | MH⁺ = 535.8 t = 1.73 MS5 |
| 102 | 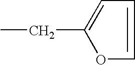 | Me | 4-Cl | 2,4-diCl | MH⁺ = 499.9 t = 1.60 MS5 |
| 103 | 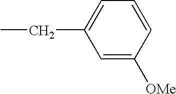 | Me | 4-Cl | 2,4-diCl | MH⁺ = 539.9 t = 1.69 MS5 |
| 104 | 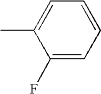 | Me | 4-Cl | 2,4-diCl | MH⁺ = 513.8 t = 1.73 MS5 |
| 105 | 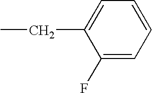 | Me | 4-Cl | 2,4-diCl | MH⁺ = 527.8 t =1.71 MS5 |
| 106 | 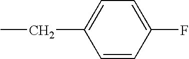 | Me | 4-Cl | 2,4-diCl | MH⁺ = 527.8 t = 1.70 MS5 |
| 107 | —Pr | Me | 4-Cl | 2,4-diCl | MH⁺ = 462.5 |
| | | | | | t = 1.57 |
| | | | | | MS2 |
| 108 | 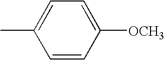 | Me | 4-Cl | 2,4-diCl | MH⁺ = 580.5 t = 1.81 MS2 |
| 109 | 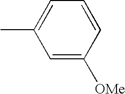 | Me | 4-Cl | 2,4-diCl | MH⁺ = 526.5 t = 1.70 MS2 |
| 110 | 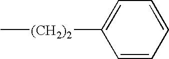 | Me | 4-Cl | 2,4-diCl | MH⁺ = 594.5 t = 1.69 MS2 |
| 111 | 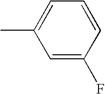 | Me | 4-Cl | 2,4-diCl | MH⁺ = 514.5 t = 1.74 MS2 |
| 112 | 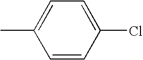 | Me | 4-Cl | 2,4-diCl | MH⁺ = 530.5 t = 1.78 MS2 |
| 113 | 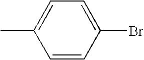 | Me | 4-Cl | 2,4-diCl | MH⁺ = 574.4 t = 1.80 MS2 |
| 114 | 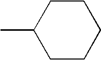 | Me | 4-OMe | 2,4-diCl | MH⁺ = 498.6 t = 1.56 MS2 |
| 115 | 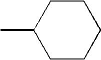 | Me | 4-Cl | 2,4-diCl | MH⁺ = 502.6 t = 1.69 MS2 |
| 116 | 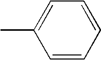 | Me | 4-Cl | 2,4-diCl | MH⁺ = 496.5 t = 1.69 MS2 |

TABLE 4

| | |
|---|---|
| | (ID) |
| 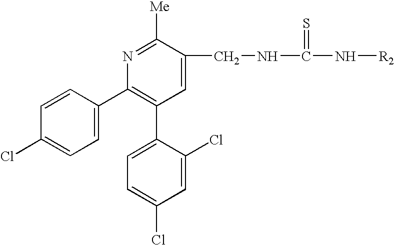 | |

| Compound | | |
|---|---|---|
| No. | R₂ | Characterization |
| 117 | 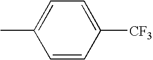 | MH⁺ = 579.8 t = 1.90 MS5 |
| | 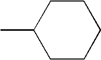 | MH⁺ = 518.5 t = 1.81 MS2 |
| | 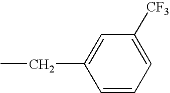 | MH⁺ = 628.8 t = 11.92 A |

TABLE 5

| | |
|---|---|
| | (IF) |
| 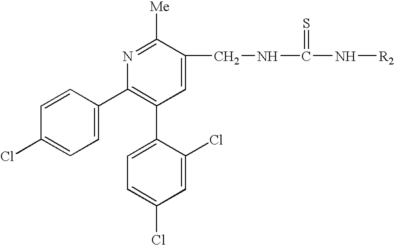 | |

| Compound | | |
|---|---|---|
| No. | R₂ | Characterization |
| 120 | 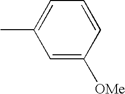 | MH⁺ = 527.0 t = 11.77 A |
| 121 | 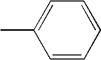 | MH⁺ = 497.0 t = 11.81 A |
| 122 | 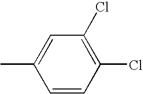 | MH⁺ = 564.8 t = 12.84 A |

The compounds according to the invention underwent pharmacological tests to determine their affinity and their antagonist power with respect to the CB₁ cannabinoid receptors.

The compounds of formula (I) have good in vitro affinity (IC₅₀≦5×10⁻⁷M) for the CB₁ cannabinoid receptors, under the experimental conditions described by M. Rinaldi-Carmona et al. (FEBS Letters, 1994, 350, 240-244).

The antagonist nature of the compounds for formula (I) was demonstrated by means of the results obtained in models of inhibition of adenylate cyclase as described in M. Rinaldi-Carmona et al., J. Pharmacol. Exp. Ther., 1996, 278, 871-878 and M. Bouaboula et al., J. Biol. Chem., 1997, 272, 22330-22339.

The toxicity of the compounds of formula (I) is compatible with their use as medicaments.

Thus, according to another of its aspects, a subject of the invention is medicaments for human or veterinary medicine, comprising a compound of formula (I) or an addition salt thereof with a pharmaceutically acceptable acid, or alternatively a solvate or a hydrate of the compound of formula (I).

The compounds according to the invention may be used in man or animals in the treatment or prevention of diseases involving the CB₁ cannabinoid receptors.

For example, and in a non-limiting manner, the compounds of formula (I) are useful as psychotropic medicaments, especially for treating psychiatric disorders including anxiety, depression, mood disorders, insomnia, delirium disorders, obsessive disorders, psychoses in general, schizophrenia, attention and hyperactivity disorders (AHD) in hyperkinetic children (MBD), and also for the treatment of disorders associated with the use of psychotropic substances, especially in the case of a substance abuse and/or dependency on a substance, including alcohol dependency and nicotine dependency.

The compounds for formula (I) according to the invention may be used as medicaments for treating migraine, stress, diseases of psychosomatic origin, panic attacks, epilepsy, motor disorders, in particular dyskinesia or Parkinson's disease, trembling and dystonia.

The compounds of formula (I) according to the invention may also be used as medicaments in the treatment of memory disorders, cognitive disorders, in particular in the treatment of senile dementia and Alzheimer's disease, and also in the treatment of attention or consciousness disorders. Furthermore, the compounds of formula (I) may be useful as neuroprotective agents, in the treatment of ischemia, cranial trauma and the treatment of neurodegenerative diseases: including chorea, Huntington's chorea and Tourrette's syndrome.

The compounds of formula (I) according to the invention may be used as medicaments in the treatment of pain, neuropathic pain, acute peripheral pain, chronic pain of inflammatory origin.

The compounds of formula (I) according to the invention may be used as medicaments in the treatment of appetite disorders, appetence disorders (for sugars, carbohydrates, drugs, alcohol or any appetizing substance) and/or eating behavioral disorders, especially for the treatment of obesity or bulimia and also for the treatment of type II diabetes or non-insulin-dependent diabetes and for the treatment of dyslipidemia and metabolic syndrome. Thus, the compounds of formula (I) according to the invention are useful in the treatment of obesity and the risks associated with obesity, especially the cardiovascular risks. Furthermore, the compounds of formula (I) according to the invention may be used as medicaments in the treatment of gastrointestinal disorders, diarrhea disorders, ulcers, vomiting, bladder and urinary disorders, disorders of endocrine origin, cardiovascular disorders, hypotension, hemorrhagic shock, septic shock, chronic cirrhosis of the liver, hepatic steatosis, steatohepatitis, chronic hepatic encephalopathy, asthma, Raynaud's syndrome, glaucoma, fertility disorders, inflammatory phenomena, immune system diseases, in particular autoimmune diseases and neuroinflammatory diseases such as rheumatoid arthritis, reactional arthritis, diseases resulting in demyelinization, multiple sclerosis, infectious and viral diseases such as encephalitis, strokes, and also as medicaments for anticancer chemotherapy, for the treatment of Guillain-Barré syndrome and for the treatment of osteoporosis.

According to the present invention, the compounds for formula (I) are most particularly useful for treating psychotic disorders, in particular schizophrenia, attention and hyperactivity disorders (AHD) in hyperkinetic children (MBD); for treating appetite and obesity disorders; for treating memory and cognitive deficits; for treating alcohol dependency and nicotine dependence, i.e. for weaning from alcohol and for weaning from tobacco; and for treating dyslipidemia and metabolic syndrome.

More particularly, the compounds of formula (I) according to the present invention are useful in the treatment and prevention of appetite disorders, metabolic disorders, gastro-intestinal disorders, inflammatory phenomena, diseases of the immune system, psychotic disorders, alcohol dependency and nicotine dependency.

According to one of its aspects, the present invention relates to the use of a compound of formula (I), pharmaceutically acceptable salts thereof and solvates or hydrates thereof for treating the disorders and diseases indicated above.

According to another of its aspects, the present invention relates to pharmaceutical compositions comprising, as active principle, a compound according to the invention. These pharmaceutical compositions contain an effective dose of at least one compound according to the invention, or a pharmaceutically acceptable salt, a solvate or a hydrate of the said compound, and also at least one pharmaceutically acceptable excipient.

The said excipients are chosen according to the pharmaceutical form and the desired mode of administration, from the usual excipients known to those skilled in the art.

In the pharmaceutical compositions of the present invention for oral, sublingual subcutaneous, intramuscular, intravenous, topical, local, intratracheal, intranasal, transdermal or rectal administration, the active principle of formula (I) above, or the possible salt, solvate or hydrate thereof, may be administered in a unit form of administration, as a mixture with standard pharmaceutical excipients, to man and animals for the prophylaxis or treatment of the above disorders or diseases.

The appropriate unit forms of administration include oral-route forms such as tablets, soft or hard gel capsules, powders, granules and oral solutions or suspensions, sublingual, buccal, intratracheal, intraocular and intranasal administration forms, forms for administration by inhalation, topical, transdermal, subcutaneous, intramuscular or intravenous administration forms, rectal administration forms and implants. For topical application, the compounds according to the invention may be used in creams, gels, pomades or lotions.

By way of example, a unit form of administration of a compound according to the invention in table form may comprise the following components:

| | |
|---|---|
| Compound according to the invention | 50.0 mg |
| Mannitol | 223.75 mg |
| Sodium croscarmellose | 6.0 mg |
| Corn starch | 15.0 mg |
| Hydroxypropylmethylcellulose | 2.25 mg |
| Magnesium stearate | 3.0 mg |

Via the oral route, the dose of active principle administered per day may be from 0.01 to 100 mg/kg in one or more dosage intakes, preferentially 0.02 to 50 mg/kg.

There may be particular cases in which higher or lower dosages are appropriate; such dosages do not depart from the context of the invention. According to the usual practice, the dosage that is appropriate to each patient is determined by the doctor according to the mode of administration and the weight and response of the said patient.

According to another of its aspects, the present invention also relates to a method for treating the pathologies indicated above, which comprises the administration to a patient of an effective dose of a compound according to the invention, or a pharmaceutically acceptable salt or hydrate or solvate thereof.

According to the present invention, a compound of formula (I) may be combined with another active principle chosen from one of the following therapeutic classes:
an angiotensin II AT₁ receptor antagonist, alone or in combination with a diuretic;
a converting enzyme inhibitor, alone or in combination with a diuretic or a calcium antagonist;
a calcium antagonist;
a beta-blocker alone or in combination with a diuretic or a calcium antagonist;
an antihyperlipidaemiant or an antihypercholesterolaemiant;
an antidiabetic agent;
another anti-obesity agent.

Thus, a subject of the present invention is also pharmaceutical compositions containing in combination a compound of formula (I) and another active principle chosen from one of the following therapeutic classes:
an angiotensin II AT₁ receptor antagonist, alone or in combination with a diuretic or a calcium antagonist;
a converting enzyme inhibitor, alone or in combination with a diuretic;
a calcium antagonist;
a beta-blocker alone or in combination with a diuretic or a calcium antagonist;
an antihyperlipidaemiant or an antihypercholesterolaemiant;
an antidiabetic agent;
another anti-obesity agent.

The term “angiotensin II AT₁ receptor antagonist” especially means a compound such as candesartan, cilexitil, eprosartan, irbesartan, losartan potassium, olmesartan medoxomil, telmisartan or valsartan, each of these compounds themselves possibly being combined with a diuretic such as hydrochlorothiazide.

The term “converting enzyme inhibitor” especially means a compound such as alacepril, benazepril, captopril, cilazapril, enalapril, enalaprilat, fosinopril, imidapril, lisinopril, moexipril, perindopril, quinapril, ramipril, spirapril, temocapril, trandolapril or zofenopril, each of these compounds itself possibly being combined with a diuretic such as hydrochlorothiazide or indapamide or with a calcium antagonist such as amlodipine, diltiazem, felodipine or verapamil.

The term “calcium antagonist” especially means a compound such as amlodipine, aranidipine, benidipine, bepridil, cilnidipine, diltiazem, efonidipine hydrochloride ethanol, fasudil, felodipine, isradipine, lacidipine, lercanidipine hydrochloride, manidipine, mibefradil hydrochloride, nicardipine, nifedipine, nilvadipine, nimodipine, nisoldipine, nitrendipine, terodiline or verapamil.

The term “beta-blocker” especially means a compound such as acebutolol, alprenolol, amosulalol, arotinolol, atenolol, befunolol, betaxolol, bevantolol, bisoprolol, bopindolol, bucumolol, bufetolol, bunitrolol, butofilolol, carazolol, carteolol, carvedilol, cloranolol, epanolol, esmolol, indenolol, labetalol, landiolol, levobunolol, levomoprolol, mepindolol, metipranolol, metoprolol, nadolol, nebivolol, nifenalol, nipradilol, oxprenolol, penbutolol, pindolol, propanolol, salmeterol, sotalol, talinolol, tertatolol, tilisolol, timolol, xamoterol or xibenolol.

The term “antihyperlipidaemiant or antihypercholesterolaemiant” especially means a compound chosen from fibrates such as alufibrate, beclobrate, bezafibrate, ciprofibrate, clinofibrate, clofibrate, etofibrate or fenofibrate; statins (HMG-CoA reductase inhibitors) such as atorvastatin, fluvastatin sodium, lovastatin, pravastatin, rosuvastatin or simvastatin, or a compound such as acipimox, aluminum nicotinate, azacosterol, cholestyramine, dextrothyroxine, meglutol, niceritrol, nicoclonate, nicotinic acid, beta-sitosterin or tiadenol. More particularly, a subject of the present invention is a pharmaceutical composition containing in combination a compound of formula (I) and atorvastatin or pravastatin or, preferentially, a compound of formula (I) and simvastatin.

The term “antidiabetic agent” especially means a compound belonging to one of the following classes: sulfonylureas, biguanidines, alpha-glucosidase inhibitors, thiazolidinediones, metiglinides such as acarbose, acetohexamide, carbutamide, chlorpropamide, glibenclamide, glibornuride, gliclazide, glimepiride, glipizide, gliquidone, glisoxepide, glybuzole, glymidine, metahexamide, metformin, miglitol, nateglinide, pioglitazone, repaglinide, rosiglitazone, tolazamide, tolbutamide, troglitazone or voglibose.

The term “another anti-obesity agent” especially means a compound such as amfepramone, benfluorex, benzphetamine, indanorex, mazindole, mefenorex, methamphetamine or D-norpseudoephedrine or another CB₁ cannabinoid receptor antagonist.

Most particularly, a subject of the present invention is a pharmaceutical composition containing in combination a compound of formula (I) and an angiotensin II AT₁ receptor antagonist, especially irbesartan, losartan or valsartan.

According to another aspect of the invention, the compound of formula (I) and the other combined active principle may be administered simultaneously, separately or sequentially over time.

The term “separate use” means the administration, at the same time, of the two compounds of the composition according to the invention, each included in a separate pharmaceutical form.

The term “use sequentially over time” means the successive administration of the first compound of the composition according to the invention, included in one pharmaceutical form, followed by the second compound of the composition according to the invention, included in a separate pharmaceutical form.

Although the invention has been illustrated by certain of the preceding examples, it is not to be construed as being limited thereby; but rather, the invention encompasses the generic area as hereinbefore disclosed. Various modifications and embodiments can be made without departing from the spirit and scope thereof.

What is claimed is:

1. A compound of the formula (I) or a salt, hydrate or solvate thereof: 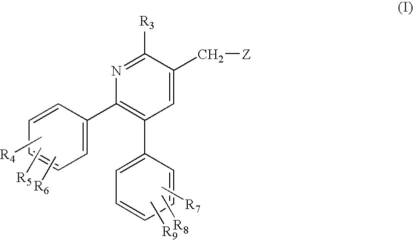
in which
Z represents a group N(R₁)XR₂, N(R₁)COOR′₂ or OCON(R₁)R′₂;
X represents a group —CO—, —SO₂—, —CON(R₁₀)— or —CSN(R₁₀)—;
R₁ represents a hydrogen atom or a (C₁-C₄)alkyl group;
R₂ represents:
a (C₃-C₁₀)alkyl group, which is unsubstituted or substituted with a CF₃ group;
a non-aromatic (C₃-C₁₂) carbocyclic radical, which is unsubstituted or substituted one or more times with identical or different substituents chosen from a (C₁-C₄)alkyl, hydroxyl, (C₁-C₄)alkoxy, (C₁-C₄)alkylthio or cyano group;
a saturated or unsaturated heterocyclic radical of 4 to 8 atoms containing oxygen, sulfur or nitrogen, which is unsubstituted or substituted with one or more identical or different substituents chosen from a halogen atom and a (C₁-C₄)alkyl, hydroxyl, trifluoromethyl, (C₁-C₄)alkoxy, trifluoromethoxy, (C₁-C₄)alkylthio, cyano or nitro group;
an indolyl group, which is unsubstituted or substituted with a halogen atom or with a (C₁-C₄)alkyl, trifluoromethyl, hydroxyl, (C₁-C₄)alkoxy, trifluoromethoxy, (C₁-C₄)alkylthio, cyano or nitro group;
a tetrahydro-1- or -2-naphthyl; a 1- or 2-naphthyl;
a benzothiophenyl or a benzofuryl;
a phenyl, which is unsubstituted or substituted one or more times with identical or different substituents chosen from a halogen atom, a (C₁-C₄)alkyl, trifluoromethyl, trifluoromethoxy, hydroxyl, (C₁-C₄)alkoxy, cyano, nitro, (C₁-C₄)alkanoyl or phenyl group and a group S(O)ₙAlk or NR₁₃R₁₄;
a benzodioxyl;
a phenoxymethyl, a 1-phenoxyethyl or a 1-methyl-1-phenoxyethyl, the phenyl groups being unsubstituted or substituted one or more times with identical or different substituents chosen from a halogen atom and a (C₁-C₄)alkyl or trifluoromethyl group;
a phenylcyclopropyl, the phenyl group being unsubstituted or substituted one or more times with identical or different substituents chosen from a halogen atom and a (C₁-C₄)alkyl or trifluoromethyl group;
a (C₁-C₂)alkylene substituted with one or two identical or different substituents chosen from:
(i) a (C₁-C₄)alkyl group;
(ii) a non-aromatic C₃ C₁₂ carbocyclic radical, which is unsubstituted or substituted one or more times with a (C₁-C₄)alkyl group;
(iii) a phenyl, which is unsubstituted or substituted with one or more substituents, which may be identical or different, chosen from a halogen atom, a (C₁-C₄)alkyl, hydroxyl, trifluoromethyl, (C₁-C₄)alkoxy, trifluoromethoxy, (C₁-C₄)alkanoyl, cyano, nitro or phenyl group and a group S(O)ₙAlk or NR₁₃R₁₄;
(iv) a saturated or unsaturated heterocyclic radical of 4 to 8 atoms, containing oxygen, sulfur or nitrogen, which is unsubstituted or substituted with one or more substituents, which may be identical or different, chosen from a halogen atom and a (C₁-C₄)alkyl or trifluoromethyl group;
with the proviso that, when X represents a group —CON(R₁₀)— or —CSN(R₁₀)—, R₂ is either a (C₁-C₆)alkanoyl group, a benzoyl or benzylcarbonyl group, the phenyl group of said groups being unsubstituted or substituted with identical or different substituents chosen from a halogen atom and a (C₁-C₄)alkyl or trifluoromethyl group;
R′₂ represents a phenyl, which is unsubstituted or substituted one or more times with identical or different substituents chosen from a halogen atom and a (C₁-C₄)alkyl, trifluoromethyl, cyano, nitro or (C₁-C₄)alkoxy group;
R₃ represents a hydrogen atom or a (C₁-C₄)alkyl, cyano, (C₁-C₄)alkoxymethyl or hydroxymethyl group;
R₄, R₅, R₆, R₇, R₈ and R₉ represent, independently of each other, a hydrogen or halogen atom, a (C₁-C₆)alkyl, (C₁-C₆)alkoxy, trifluoromethyl, trifluoromethoxy, cyano or nitro group or a group S(O)ₙAlk;
R₁₀ represents a hydrogen atom or a (C₁-C₄)alkyl group;
or R₂ and R₁₀, together with the nitrogen atom to which they are attached, constitute a heterocyclic radical of 4 to 8 atoms, optionally containing a second heteroatom chosen from an oxygen, a sulfur and a nitrogen atom, which is unsubstituted or substituted one or more times with a (C₁-C₄)alkyl group; a (C₁-C₄)alkanoyl group; a group NR₁₁R₁₂ or CONR₁₁R₁₂; a phenyl group, which is unsubstituted or substituted one or more times with a halogen atom or a (C₁-C₄)alkyl, (C₁-C₄)alkoxy or trifluoromethyl group;
R₁₁ and R₁₂ represent, independently of each other, a hydrogen atom or a (C₁-C₄)alkyl group, or R₁₁ and R₁₂, together with the nitrogen atom to which they are attached, constitute a heterocyclic radical of 4 to 8 atoms;
n represents 0, 1 or 2;
R₁₃ and R₁₄ represent, independently of each other, a hydrogen atom or a (C₁-C₄)alkyl group, or R₁₃ and R₁₄, together with the nitrogen atom to which they are attached, constitute a saturated or unsaturated heterocyclic radical of 4 to 8 atoms;
Alk represents a (C₁-C₄)alkyl group; and
with the proviso that one of the substituents R₁, R₃, R₅, R₆, R₈ and R₉ is other than hydrogen when R₄ and R₇ simultaneously represent a 4-methoxy group.

2. The compound of formula (I) according to claim 1 in which Z represents a group N(R₁)XR₂, and wherein X represents a —CO— group; or a salt, a hydrate or a solvate thereof.

3. The compound of formula (I) according to claim 1 in which Z represents a group N(R₁)XR₂, and wherein X represents an —SO₂— group; or a salt, a hydrate or a solvate thereof.

4. The compound of formula (I) according to claim 1 in which Z represents a group N(R₁)XR₂, and wherein X represents a group —CON(R₁₀)—; or a salt, a hydrate or a solvate thereof.

5. The compound of formula (I) according to claim 1 in which Z represents a group N(R₁)XR₂, and wherein X represents a group —CSNR₁₀; or a salt, a hydrate or a solvate thereof.

6. The compound of formula (I) according to claim 1 in which Z represents a group N(R₁)COOR′₂; or a salt, a hydrate or a solvate thereof.

7. The compound of formula (I) according to claim 1 in which Z represents a group OCO(R₁)R′₂; or a salt, a hydrate or a solvate thereof.

8. The compound of formula (I) according to claim 1 in which:
Z represents a group N(R₁)XR₂, wherein X is as defined in claim 1;
R₁ represents a hydrogen atom;
R₂ represents a 1-propylbutyl or a 2-indolyl, which is unsubstituted or substituted with a (C₁-C₄)alkyl group, or R₂ represents a phenyl, which is unsubstituted or substituted one or more times with identical or different substituents chosen from a halogen atom and a (C₁-C₄)alkyl, trifluoromethyl, (C₁-C₄)alkoxy, cyano or phenyl group;
R₃ represents a methyl or methoxymethyl group;
R₄ represents a chlorine or bromine atom or a methoxy group;
R₇ and R₈ each represent a chlorine atom; and
R₅, R₆ and R₉ represent hydrogen;
or a salt, a hydrate or a solvate thereof.

9. The compound of formula (I) according to claim 1 in which:
Z represents a group NHCOR₂;
R₂ represents a 1-propylbutyl group, an indolyl group, which is unsubstituted or substituted with a (C₁-C₄)alkyl group, a phenyl group, which is unsubstituted or substituted with a halogen atom or a methoxy group;
R₃ represents a methyl or methoxymethyl group;
R₄ represents a chlorine or bromine atom or a methoxy group;
R₇ and R₈ each represent a chlorine atom; and
R₅, R₆ and R₉ represent hydrogen;
or a salt, a hydrate or a solvate thereof.

10. The compound of formula (I) according to claim 1 in which:
Z represents a group NHSO₂R₂;
R₂ represents a phenyl group, which is unsubstituted or substituted with a halogen atom or with a trifluoromethyl group;
R₃ represents a methyl or methoxymethyl group;
R₄ represents a chlorine or bromine atom or a methoxy group;
R₇ and R₈ each represent a chlorine atom; and
R₅, R₆ and R₉ represent hydrogen;
or a salt, a hydrate or a solvate thereof.

11. The compound of formula (I) according to claim 1, chosen from:
N-{[-6-(4-chlorophenyl)-5-(2,4-dichlorophenyl)-2-methylpyridin-3-yl]methyl}-1H-indole-2-carboxamide;
N-{[-6-(4-chlorophenyl)-5-(2,4-dichlorophenyl)-2-methylpyridin-3-yl]methyl}-4-(trifluoromethyl)benzenesulfonamide;
N-{[-6-(4-chlorophenyl)-5-(2,4-dichlorophenyl)-2-methylpyridin-3-yl]methyl}-N′-[4-(trifluoromethyl)phenyl]urea;
N-{[5-(2,4-dichlorophenyl)-2-(methoxymethyl)-6-(4-methoxyphenyl)pyridin-3-yl]methyl}-2-propylpentanamide;
N-{[5-(2,4-dichlorophenyl)-2-(methoxymethyl)-6-(4-methoxyphenyl)pyridin-3-yl]methyl}-1H-indole-2-carboxamide;
N-{[6-(4-chlorophenyl)-5-(2,4-dichlorophenyl)-2-(methoxymethyl)pyridin-3-yl]methyl}-4-(trifluoromethoxy)benzamide; and
N-{[6-(4-chlorophenyl)-5-(2,4-dichlorophenyl)-2-(methoxymethyl)pyridin-3-yl]methyl}-2-propylpentanamide;
or a salt, a hydrate or a solvate thereof.

12. A process for preparing a compound of formula (I) according to claim 1, comprising:
treating a compound of formula (II): 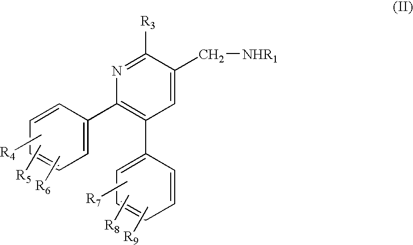
in which R₁ and R₃ to R₉ are as defined in claim 1;
either with an acid of formula R₂CO₂H (III) in which R₂ is as defined in claim 1, or with an activated derivative of said acid, when a compound of formula (IA) in which X represents a —CO-group is prepared;
or with a sulfonyl halide of formula R₂SO₂Hal (IV) in which R₂ is as defined in claim 1 and Hal represents a halogen atom, when a compound of formula (IB) in which X represents an —SO₂-group is prepared.

13. A process for preparing a compound of formula (I) according to claim 1 in which Z represents a group O—CO—NH—R′₂, comprising:
treating a compound of formula (IX): 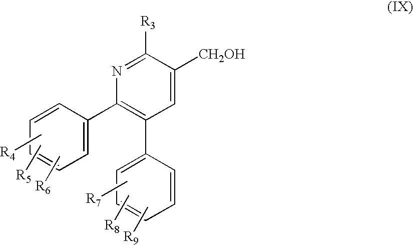
in which R₃ to R₉ are as defined in claim 1;
with a compound of formula R′₂—N═C═O, in which R′₂ is as defined in claim 1.

14. A compound of formula (II): 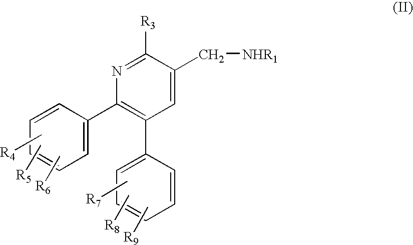
in which:
R₁ represents a hydrogen atom or a (C₁-C₄)alkyl group;
R₃ represents a hydrogen atom or a (C₁-C₄)alkyl, cyano, (C₁-C₄)alkoxymethylene or hydroxymethyl group;
R₄, R₅, R₆, R₇, R₈ and R₉ represent, independently of each other, a hydrogen or halogen atom, a (C₁-C₆)alkyl, (C₁-C₆)alkoxy, trifluoromethyl, trifluoromethoxy, cyano or nitro group or a group S(O)ₙAlk;
with the proviso that one of the substituents R₁, R₃, R₅, R₆, R₈ and R₉ is other than hydrogen when R₄ and R₇ simultaneously represent a 4-methoxy group.

15. A pharmaceutical composition comprising a compound of formula (I) according to claim 1, or a pharmaceutically acceptable salt, a hydrate or a solvate thereof in combination with at least one pharmaceutically acceptable excipient.

16. A pharmaceutical composition comprising a compound of formula (I) according to claim 2, or a pharmaceutically acceptable salt, a hydrate or a solvate thereof in combination with at least one pharmaceutically acceptable excipient.

17. A pharmaceutical composition comprising a compound of formula (I) according to claim 3, or a pharmaceutically acceptable salt, a hydrate or a solvate thereof in combination with at least one pharmaceutically acceptable excipient.

18. A pharmaceutical composition comprising a compound of formula (I) according to claim 4, or a pharmaceutically acceptable salt, a hydrate or a solvate thereof in combination with at least one pharmaceutically acceptable excipient.

19. A pharmaceutical composition comprising a compound of formula (I) according to claim 5, or a pharmaceutically acceptable salt, a hydrate or a solvate thereof in combination with at least one pharmaceutically acceptable excipient.

20. A pharmaceutical composition comprising a compound of formula (I) according to claim 6, or a pharmaceutically acceptable salt, a hydrate or a solvate thereof in combination with at least one pharmaceutically acceptable excipient.

21. A pharmaceutical composition comprising a compound of formula (I) according to claim 7, or a pharmaceutically acceptable salt, a hydrate or a solvate thereof in combination with at least one pharmaceutically acceptable excipient.

22. A pharmaceutical composition comprising a compound of formula (I) according to claim 8, or a pharmaceutically acceptable salt, a hydrate or a solvate thereof in combination with at least one pharmaceutically acceptable excipient.

23. A pharmaceutical composition comprising a compound of formula (I) according to claim 9, or a pharmaceutically acceptable salt, a hydrate or a solvate thereof in combination with at least one pharmaceutically acceptable excipient.

24. A pharmaceutical composition comprising a compound of formula (I) according to claim 10, or a pharmaceutically acceptable salt, a hydrate or a solvate thereof in combination with at least one pharmaceutically acceptable excipient.

25. A pharmaceutical composition comprising a compound of formula (I) according to claim 11, or a pharmaceutically acceptable salt, a hydrate or a solvate thereof in combination with at least one pharmaceutically acceptable excipient.

26. A method for treating appetite disorders, metabolic disorders, gastrointestinal disorders, inflammatory phenomena, immune system diseases, psychotic disorders, alcohol dependency and nicotine dependency in a patient, which comprises administering to said patient an effective amount of a compound of formula (I) according to claim 1.

27. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 2.

28. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 3.

29. A method for treating appetite disorders, metabolic disorders, gastrointestinal disorders, inflammatory phenomena, immune system diseases, psychotic disorders, alcohol dependency and nicotine dependency in a patient, which comprises administering to said patient an effective amount of a compound of formula (I) according to claim 4.

30. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 5.

31. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 6.

32. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 7.

33. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 8.

34. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 9.

35. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 10.

36. A method for treating appetite disorders, metabolic disorders, gastro-intestinal disorders, inflammatory phenomena, immune system diseases, psychotic disorders, alcohol dependency and nicotine dependency in a patient, which comprises administering to said patient an effective amount of a compound of formula (I) according to claim 11.
